SEQUENCE LISTING (17 sequences)

SEQ ID NO: 1 - DNA, 21 bp, Artificial
  pXN2 backbone
ctaacagatatcacgctcgag

SEQ ID NO: 2 - DNA, 1587 bp, Artificial
  Single chain IL23
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggcggtggctcgggcggtggtgggtcgggtggcggcggatccctggctgtgcctaggagtagcagtcctgactgggctcagtgccagcagctctctcggaatctctgcatgctagcctggaacgcacatgcaccagcgggacatatgaatctactaagagaagaagaggatgaagagactaaaaataatgtgccccgtatccagtgtgaagatggttgtgacccacaaggactcaaggacaacagccagttctgcttgcaaaggatccgccaaggtctggctttttataagcacctgcttgactctgacatcttcaaaggggagcctgctctactccctgatagccccatggagcaacttcacacctccctactaggactcagccaactcctccagccagaggatcacccccgggagacccaacagatgcccagcctgagttctagtcagcagtggcagcgcccccttctccgttccaagatccttcgaagcctccaggcctttttggccatagctgcccgggtctttgcccacggagcagcaactctgactgagcccttagtgccaacagctactagc

SEQ ID NO: 3 - DNA, 90 bp, Artificial
  pXN2 backbone with stop codon and poly A tail
agattcttcatgtttggaccaaatcaacttgtgataccatgctcaaagaggcctcaattatatttgagtttttaatttttatgaaaaaaa

SEQ ID NO: 4 - DNA, 1581 bp, Artificial
  Single chain IL23 with stop mutations
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgatgatgactggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggcggtggctcgggcggtggtgggtcgggtggcggcggatccctggctgtgcctaggagtagcagtcctgactgggctcagtgccagcagctctctcggaatctctgcatgctagcctggaacgcacatgcaccagcgggacatatgaatctactaagagaagaagaggatgaagagactaaaaataatgtgccccgtatccagtgtgaagatggttgtgacccacaaggactcaaggacaacagccagttctgcttgcaaaggatccgccaaggtctggctttttataagcacctgcttgactctgacatcttcaaaggggagcctgctctactccctgatagccccatggagcaacttcacacctccctactaggactcagccaactcctccagccagaggatcacccccgggagacccaacagatgcccagcctgagttctagtcagcagtggcagcgcccccttctccgttccaagatccttcgaagcctccaggcctttttggccatagctgcccgggtctttgcccacggagcagcaactctgactgagcccttagtgccaacagct

SEQ ID NO: 5 - DNA, 42 bp, Artificial
  Target region of pXN2-scIL23 sequence
ctaacagatatcacgctcgagatgtgtcctcagaagctaacc

SEQ ID NO: 6 - DNA, 6 bp, Artificial
  NruI sequence
tcgcga

SEQ ID NO: 7 - DNA, 42 bp, Artificial
  pXN2-Nru-scIL23 sequence
cttcgcgatatcacgctcgagatgtgtcctcagaagctaacc

SEQ ID NO: 8 - DNA, 20 bp, Artificial
  JM005 IFN gamma
gctttgcagctcttcctcat

SEQ ID NO: 9 - DNA, 20 bp, Artificial
  JM006 IFN gamma
tgagctcattgaatgcttgg

SEQ ID NO: 10 - DNA, 20 bp, Artificial
  JM017 TNF alpha
gaactggcagaagaggcact

SEQ ID NO: 11 - DNA, 20 bp, Artificial
  JM01 8 TNF alpha
cggactccgcaaagtctaag

SEQ ID NO: 12 - DNA, 20 bp, Artificial
  JM01 9 Beta-Actin
aagagctatgagctgcctga

SEQ ID NO: 13 - DNA, 20 bp, Artificial
  JM020 Beta-Actin
tacggatgtcaacgtcacac

SEQ ID NO: 14 - DNA, 36 bp, Artificial
  J Mp40XhoI
tagtcctcgagatgtgtcctcagaagctaaccatct

SEQ ID NO: 15 - DNA, 34 bp, Artificial
  JMp1 9SpeI
tatgaactagtctaagctgttggcactaagggct

SEQ ID NO: 16 - DNA, 45 bp, Artificial
  J M033p40MutF
actccggacggttcacgtgatgatgactggtgcaaagaaacatgg

SEQ ID NO: 17 - DNA, 45 bp, Artificial
  J M034p40MutR
ccatgtttctttgcaccagtcatcatcacgtgaaccgtccggagt

(19) United States
(12) Patent Application Publication
(10) Pub. No.: US 2011/0014228 A1
(43) Pub. Date: Jan. 20, 2011

(54) IL23 MODIFIED VIRAL VECTOR FOR RECOMBINANT VACCINES AND TUMOR TREATMENT

(75) Inventors: Carol Shoshkes REISS, New York, NY (US); James M. MILLER, New York, NY (US)

Correspondence Address: NIXON PEABODY LLP - PATENT GROUP, 1100 CLINTON SQUARE, ROCHESTER, NY, 14604, US

(73) Assignee: NEW YORK UNIVERSITY, New York, NY (US)

(21) Appl. No.: 12/816,024

(22) Filed: Jun. 15, 2010

Related U.S. Application Data
(60) Provisional application No. 61/187,125, filed on Jun. 15, 2009.

(51) Int. Cl. A61K 39/12; C12N 7/01; C07H 21/02; C12N 5/10; A61K 35/76; A61P 35/00; A61P 37/04

(52) U.S. Cl. 424/204.1; 435/235.1; 536/23.5; 435/325; 536/23.72; 424/93.6

(57) ABSTRACT

The present invention relates to recombinant replicable viral vectors and viruses which are modified with IL23. This IL23 modified virus is highly immunogenic and attenuated for neurotropic pathology found in the wild type viruses. These viruses and vectors can be used for treatment of a variety of cancers and for vaccination against many viral, bacterial, or parasitic diseases.

This application claims the benefit of U.S. Provisional Patent Application Ser. No. 61/187,125, filed Jun. 15, 2009, which is hereby incorporated by reference in its entirety.

This invention was made with government support under grant number R01NS039746 awarded by the National Institute of Neurological Diseases and Stroke of the National Institutes of Health. The government has certain rights in this invention.

### FIELD OF THE INVENTION

The present invention relates to IL23 modified viral vectors and viruses that can be used for making vaccines and for treating cancer.

### BACKGROUND OF THE INVENTION

Rhabdoviruses, belonging to the family Rhabdoviridiae, are membrane enveloped viruses shaped like a rod. They infect a range of hosts throughout the animal and plant kingdom. Rhabdoviruses have a negative-sense single stranded RNA genome that has around 11,000-12,000 nucleotides (Rose et al. “Rhabdovirus Genomes and their Products,” in The Viruses: The Rhabdoviruses, Plenum Publishing Corp). Typically the genome codes for five proteins, three out of five namely large protein (L), nucleoprotein (N) and phosphoprotein (P) are found associated with the viral genome. The other two are glycoprotein (G), which forms spikes on the surface of the virus particle, and matrix protein (M) which lies within the membrane envelope. Rhabdoviruses must encode for a RNA-dependent RNA polymerase because the genome is a negative sense RNA and must be transcribed into a positive sense mRNA so that it can later be translated into viral proteins (Baltimore et al., “Ribonucleic Acid Synthesis of Vesicular Stomatitis Virus, II. An RNA Polymerase in the Virion,” Proc Nat'l Acad. Sci. USA 66:572-576 (1970)). Proteins L and P make the RNA-dependent RNA polymerase and also regulate the transcription process. Replication of many Rhabdoviruses occurs in the cytoplasm except several of the plant infecting viruses where the replication takes place in the nucleus.

There are two distinct genera within the Rhabdoviridiae family, the Lyssavirus and the Vesiculovirus. Vesicular stomatitis virus (VSV), a prototypical member of the genus Vesiculovirus, is a naturally occurring virus which is transmitted by sand-flies to cattle, and causes the eponymous small oral rashes. The VSV genome has a negative sense genome, which is complementary to the positive sense mRNA that encodes proteins. The sequences of the VSV mRNAs and genome is described in Gallione et al., “Nucleotide Sequences of the mRNA's Encoding the Vesicular Stomatitis Virus N and NS Proteins,” J. Virol. 39(2):529-35 (1981) and Rose et al., “Nucleotide Sequences of the mRNA's Encoding the Vesicular Stomatitis Virus G and M Proteins Determined from cDNA Clones Containing the Complete Coding Regions,” J. Virol. 39(2):519-28 (1981). VSV rarely infects humans but when an infection occurs it can remain asymptomatic or cause mild flu like symptoms (Fields et al., “Human Infection with the Virus of Vesicular Stomatitis During an Epizootic,” N. Engl. J. Med. 277:989-994 (1967); Johnson et al., “Clinical and Serological Response to Laboratory-acquired Human Infection by Indiana Type Vesicular Stomatitis Virus (VSV),” Am. J. Trop. Med. Hyg. 15:244-246 (1966)).

Vesicular stomatitis virus (VSV) has potential uses as a live attenuated viral vector for vaccination or as an oncolytic vector. VSV also has the ability to selectively target tumor cells which have lost their interferon responsiveness (Balachandran et al., “Defective Translational Control Facilitates Vesicular Stomatitis Virus Oncolysis,” Cancer Cell 5:51-65 (2004)). Interferons induced by a VSV infection protects normal tissue from the virus whereas VSV rapidly replicates and selectively kills a variety of human tumor cell lines which have compromised interferon pathways (Barber, “Vesicular Stomatitis Virus as an Oncolytic Vector,” Viral Immunol. 17(4):516-27 (2004); Stodjl et al., “Exploiting Tumor-specific Defects in the Interferon Pathway with a Previously Unknown Oncolytic Virus,” Nature Medicine 6:821-825 (2000)). VSV can also be used as a viral vector for vaccination. The use of recombinant VSV-based vectors can be an effective and promising platform for the development of preventive vaccines against a number of pathogenic organisms and diseases. The advantages of live attenuated virus vaccines are their capacity of replication and induction of both humoral and cellular immune responses. Also, there is low degree of seropositivity in general population against VSV and in general live attenuated viruses have longer lasting immunity after a single administration. However, safety is an extremely important concern when using live attenuated viruses. The virus should have the ability to induce an immune response without causing pathology in the subject. This is an important concern when using VSV as a therapeutic agent because VSV can be highly neurotropic.

Studies have shown that VSV in many cases can potentially cause an unacceptable side-effect of viral encephalitis. The vesicular stomatitis virus (VSV) causes severe central nervous system (CNS) pathology when administered to mice intranasally (i.n.) (Sabin et al., “Influence of Host Factors on Neuroinvasiveness of Vesicular Stomatitis Virus: III. Effect of Age and Pathway of Infection on the Character and Localization of Lesions in the Central Nervous System,” J Exp Med 67:201-228 (1938); Huneycutt et al., “Distribution of Vesicular Stomatitis Virus Proteins in the Brains of BALB/c Mice Following Intranasal Inoculation: An Immunohistochemical Analysis,” Brain Res 635(1-2):81-95 (1994)). Immunocompetent mice exhibit high morbidity and mortality at low doses of virus, succumbing to infection between 6 and 11 days post infection (p.i.). In contrast, inoculation of immunocompetent mice with high doses of VSV by the intramuscular, subcutaneous, or intraperitoneal routes generally leads to limited viral replication and no apparent disease (Huneycutt et al., “Distribution of Vesicular Stomatitis Virus Proteins in the Brains of BALB/c Mice Following Intranasal Inoculation: An Immunohistochemical Analysis,” Brain Res 635:81 (1994)). Similarly, intravenous (i.v.) inoculation of mice with high doses of VSV leads to limited viral replication in the periphery, but can cause CNS pathology if virus gains access to the brain. A recent study in non human primates demonstrated significant neuropathology following intrathalamic inoculation of cynomolgus macaques (Johnson et al., “Neurovirulence Properties of Recombinant Vesicular Stomatitis Virus Vectors in Non-human Primates,” Virology 360:36-49 (2007)). The pathology of VSV necessitates the use of attenuated virus when used as a therapeutic. However, there are certain factors that need to be considered when using attenuation as a means to eliminate the chance of pathogenesis by a virus.

In general, viruses are attenuated or killed when used for vaccination or as a therapeutic. A major concern with the attenuation is the risk of reversion to virulence (Ruprecht, “Live Attenuated AIDS Viruses as Vaccines: Promise or Peril?” Immunol Rev. 170:135-49 (1999); Minor, “Attenuation and Reversion of the Sabin Vaccine Strains of Poliovirus,” Dev. Biol. Stand. 78:17-26 (1993)) and/or insufficient attenuation/killing of a live vaccine. Further, the inactivation or attenuation, which makes the virus safer, may alter the antigens thereby making them less immunogenic and thus less effective. The key issue is to balance the safety and immunogenicity of an attenuated or inactivated virus, such that the exposure of a host to attenuated viruses would elicit a potent immune response or oncolysis. Often times it is desirable that the viruses remain replication competent. Therefore, there is a need for safe and effective attenuation of VSV in order to minimize the risks associated with pathogenesis without jeopardizing its therapeutic potential.

The present invention is directed at overcoming these and other deficiencies in the art.

### SUMMARY OF THE INVENTION

One aspect of the present invention is directed to a modified recombinant replicable vesiculovirus comprising vesiculovirus N, P, L proteins, and a replicable vesiculovirus genomic sense (−) RNA comprising a nucleic acid molecule encoding for IL23.

Another aspect of the present invention is directed to a method of treating cancer in a subject. This method involves selecting a subject with cancer and administering to the subject the recombinant replicable vesiculovirus modified with IL23 under conditions effective to treat cancer.

In another aspect, the present invention relates to a method for treating or preventing a disease or disorder mediated by a peptide or protein. This method involves selecting a subject in need of treatment or prevention of the disease or disorder. The IL23 modified recombinant vesiculovirus or vector is administered to the selected subject under conditions effective to induce an immune response against the pathogenic peptide or protein.

Another aspect of the present invention is directed to a recombinant, replicating and infectious vesicular stomatitis virus (VSV) particle which comprises a functional RNA dependent RNA polymerase (L), a vesiculovirus phosphoprotein (P), a vesiculovirus nucleocapsid (N), vesiculovirus protein selected from the group consisting of glycoprotein (G) and matrix (M), a 3′ non-coding RNA sequence, and a 3′ to 5′ RNA coding sequence, which encodes the vesiculovirus L, P, N and a vesiculovirus protein required for assembly of budded infectious particles, including a nucleic acid molecule which encodes for IL23 protein inserted at an intergenic junction, and a 5′ non-coding RNA sequence. These components are from the same type of VSV.

The present invention relates to a highly attenuated recombinant vesiculoviruses which includes an immuno-modulatory molecule Interleukin-23 (IL23). IL23 is a heterodimeric cytokine with two subunits, one called p40, which is shared with another cytokine, IL-12, and another called p19, the IL23 alpha subunit (Lankford et al., “A Unique Role for IL23 in Promoting Cellular Immunity,” J. Leukoc. Biol. 73:49-56 (2003), which is hereby incorporated by reference in its entirety). IL23 is an important part of the inflammatory response against infection, and it enhances host's innate and adaptive immune responses to the virus. VSV modified with IL23 does not cause the morbidity and mortality as seen in mice which are administered with wild type VSV or other recombinant VSV variants. Because of this loss of pathogenicity, this IL23 modified VSV can be used as a potent vaccine vector to deliver virtually unlimited pathogen proteins using a simple recombinant DNA technology and can also be used for oncolysis of tumor cells which have compromised interferon pathways.

The present invention is directed towards novel vesiculoviruses and vectors which comprises a nucleic acid encoding for IL23, a cytokine. Vesicular stomatitis virus (VSV) is a virus with a negative (−) sense RNA as the genome comprising only 5 genes encoding for proteins. Expression of IL23 leads to attenuation of VSV when introduced intranasally to mice. This modified virus is highly immunogenic and induces apoptosis in tumor cells. Because of the attenuation this modified virus can be effectively used as a vector for vaccination and for treatment of tumor cells.

### BRIEF DESCRIPTION OF THE DRAWINGS

FIGS. 1A-C show the plasmids that were used in the present invention. FIG. 1A shows the plasmid map for pXN2, FIG. 1B shows the plasmid map for pXN2-IL23, and FIG. 1C shows the plasmid map for pXN2-IL23ST. Plasmids are 16195 base pairs (bp) in length. The scIL23 (IL23) is located between the G and L protein coding regions. In the pXN2-IL23ST plasmid, stop codons are located in the p40 subunit of the IL23 region, the first at position 7979. pXN2-IL23 and pXN2-IL23ST were used to produce VSV23 and VSVST, respectively.

FIGS. 2A-C are the nucleotide sequences of VSV23 and the mutations introduced into VSV23 to generate VSVST, and modification introduced in VSV23 by creation of a novel Nru I site. FIG. 2A shows the 5′ end partial backbone sequence of plasmid pXN2 (SEQ ID NO: 1), scIL23 sequence (SEQ ID NO: 2), and 3′ end partial sequence of plasmid pXN2 backbone (SEQ ID NO: 3). The scIL23 sequence was ligated into the pXN2 backbone. The XhoI restriction site in the pXN2 backbone is highlighted in red (SEQ ID NO: 1). Start and stop codons for the scIL23 coding region are indicated by bold and blue highlighted text (SEQ ID NO: 2 and 3, respectively). FIG. 2B shows scIL23 sequence with stop mutations (SEQ ID NO: 4). The stop mutations are highlighted in blue, the altered nucleotides represented in capital letters. FIG. 2C shows the point mutations in pXN2-scIL23 that result in a unique Nru I restriction site (SEQ ID NO: 5). The region of the sequence to be mutated is highlighted in blue and the start codon (atg) of scIL23 is indicated in bold. Upstream of this site is the G protein coding region. The QuikChange® XL Site-Directed Mutagenesis Kit from Strategene can be used to induce 4 point mutations in the DNA upstream of the Xho I site resulting in a Nru I sequence highlighted in yellow (SEQ ID NO: 6). The partial sequence of the mutated plasmid (designated pXN2-Nru-scIL23) is shown with capitalized text to indicate the point mutations (SEQ ID NO: 7).

FIG. 3 shows the production of vIL23 by VSV23 infected BHK21 cells. BHK21 cells were infected with VSV23, VSVST, or VSVXN2 at MOI=0.1 and incubated overnight at 37° C.

FIGS. 4A-D shows NB41A3 cell infected with recombinant VSVs (rVSVs) and IL23. VSV23, VSVST, and VSVXN2 were used to infect NB41A3 cells at an MOI=0.001 in duplicate. Half of the samples were treated with rIL-23. Supernatant was harvested at 12 hours (FIG. 4A), 16 hours (FIG. 4B), 20 hours (FIG. 4C), and 24 hours (FIG. 4D) and stored at −80° C. Virally infected supernatants were serially diluted and transferred to fresh L929 cells for plaques assays. Results indicate that IL-23 induces a modest effect on viral titers in infected NB41A3 cells.

FIGS. 5A-D show L929 cell infection with rVSVs and IL23. VSV23, VSVST, and VSVXN2 were used to infect L929 cells at an MOI=0.001 in duplicate. Half of the samples were treated with rIL23. Supernatant was harvested at 12 hours (FIG. 5A), 16 hours (FIG. 5B), 20 hours (FIG. 5C), and 24 hours (FIG. 5D) and stored at −80° C. Virally infected supernatants were serially diluted and transferred to fresh L929 cells for plaques assays. Results indicate that IL-23 does not induce an effect on viral titers in infected L929 cells.

FIGS. 6A-B show rVSV intranasal infection morbidity data. Weight of infected mouse is shown in the FIG. 6A and a quantification of the clinical symptoms is shown in FIG. 6B. Cohorts of 9 (VSV23) or 10 (other viruses), 6-week old BALB/cAnTac mice were infected intranasally with 1×10⁴ pfu of VSV23 (blue), VSVST (pink), VSVXN2 (gold), or VSVwt (aqua) and monitored for 15 days. Mice were weighed and scored daily to assess clinical symptoms: “1” for lack of grooming behavior, “2” for hunched and severely lethargic mice, “3” for hind-limb paralysis and “4” for full paralysis or death. Hind-limb paralysis or with a weight loss of more than 25% was considered an endpoint for the experiment. Each data point represents the average score of the cohort. ANOVA analysis indicates a significant attenuation of VSV23 compared to all other VSVs; p<0.05.

FIG. 7 shows that rVSV23 infection is highly attenuated for lethal intranasal infection resulting in viral encephalitis. Cohorts of either 10 or 9,6-week old BALB/cAnTac mice were infected intranasally with 1×10⁴ pfu of VSV23 (blue), VSVST (pink), VSVXN2 (gold), or VSVwt (aqua) and monitored for 15 days. Mice were weighed daily to monitor for weight loss and if loss exceeded 25%, the NYU IACUC required humane sacrifice. VSVwt infection resulted in 70% mortality, infection with either VSVST or VSVXN2 resulted in 20% mortality, while VSV23 infection was highly attenuated and resulted in no deaths. The data for one of two representative infection studies is shown; no mice infected with VSV23 died in the other study. VSV23 is different from the other viruses by 0<0.05 in Kaplan Meier analysis.

FIG. 8 shows that rVSVs induce nitric oxide production in CNS. Cohorts of 6, 6 week old male BALB/cAnTac mice were infected intranasally with 1×10³ pfu of VSV23 (blue), VSVST (pink), VSVXN2 (gold), or mock infected (red). Brains were harvested on days 1, 3, 6, and 9 post-infection, divided into hemispheres sagitally, and half-brains were homogenized on ice. Samples were pre-cleared of solid material by centrifugation. The Total Nitric Oxide Assay Kit from Pierce was used as per manufacturer's instructions to convert nitrate to nitrite from individual homogenate samples. Equal volumes of experimental sample and Greiss reagent (1% sulfanilamide, 0.1% N-1-naphthylethylene-diamine, and 5% H₃PO₄; Sigma Chemical Co.) were incubated at room temperature for 10 min and results were read at 540 nm. VSV23 induces greater amounts of NO compared to other rVSVs and VSVwt and does so at earlier time points. Data shown are mean+/−standard deviations on days 1, 3, 6, and 9, respectively. ANOVA analysis of days 3 and 6 data reject the null hypothesis with p<0.0001, indicating that VSV23 induces significantly more NO production. Data shown are from one of 3 comparable replicate experiments.

FIG. 9 shows that rVSV23 infection is highly attenuated for lethal intranasal infection resulting in viral encephalitis. Cohorts of 20 or 19, 6-week old BALB/c mice were infected intranasally with 1×10⁶ pfu of VSV23 (blue), VSVST (pink), or VSVXN2 (yellow) and monitored for 15 days. VSVST infection resulted in 40% mortality while VSVXN2 infection resulted in 58% mortality. VSV23 infection resulted in 25% mortality. VSV23 is different from the other viruses by p<0.05 in Kaplan Meier analysis.

FIGS. 10A-B show rVSV intranasal infection morbidity. FIG. 10A shows clinical symptoms and FIG. 10B shows percent weight loss. Cohorts of 20 or 19, 6-week old BALB/c mice were infected intranasally with 1×10⁶ pfu of VSV23 (blue), VSVST (pink), or VSVXN2 (yellow) and monitored for 15 days. Mice were weighed and scored daily to assess clinical symptoms: “0” for no symptoms, “1” for lack of grooming behavior, “2” for hunched and severely lethargic mice, “3” for hind-limb paralysis and “4” for full paralysis, and “5” for death. Hind-limb paralysis or with a weight loss of more than 30% was considered an endpoint for the experiment. Each data point represents the average score of the cohort. ANOVA analysis of clinical scores indicates a significant attenuation of VSV23 compared to all other VSVs; p<0.05. Weights were comparable for all infection groups.

FIGS. 11A-B show rVSV viral titers in the CNS. Cohorts of 6 week old male BALB/c mice were infected i.n. with 1×10⁶ pfu of VSV23, VSVST, or VSVXN2. Brains were harvested on days 1 (FIG. 11A) and 3 (FIG. 11B) p.i., hemisphered sagitally, and homogenized. Samples were serially diluted and plated on L929 cells. Plaque assays were conducted to determine viral titers. Data points represent titers in individual mice. Horizontal bars indicate the geometric mean titer of the cohorts. Viral titers were similar for all infections at both time points.

FIG. 12 shows rVSVs induce nitric oxide production in CNS. Cohorts of 6, 6 week old male BALB/c mice were infected i.n. with 1×10⁶ pfu of VSV23 (blue), VSVST (pink), or VSVXN2 (yellow). Brains from individuals in each treatment group were harvested on days 1, and 3 post-infection. VSV23 induces greater amounts of NO compared to VSVST and VSVXN2. ANOVA analysis of day 3 data reject the null hypothesis with p<0.05, indicating that VSV23 induces significantly more NO production.

FIG. 13 shows that NK Cells are active in all viral treatment groups. Cohorts of 6, 6 week old male BALB/cAnTac mice were inoculated intraperitoneally with 1×10⁷ pfu of VSV23 (blue), VSVST (pink), VSVXN2 (gold), VSVwt (aqua), or mock-infected with diluent (grey). Uninfected animals were used as a negative control (red). Splenocytes were harvested 3 days post-immunization, serially diluted, and coincubated in triplicate with YAC-1 cells at 37° C. for 4 h. NK cytolytic activity was determined by using the CytoTox 96® Non-Radioactive Cytotoxicity Assay kit from Promega. All samples from virally inoculated animals showed similar levels of NK mediated cell killing. The assay shown is one of two replicate experiments with comparable results.

FIGS. 14A-B show that all virus-immune T cell populations exhibit T cell proliferation when cultured with infected stimulators. Cohorts of 6, 6 week old male BALB/cAnTac mice were inoculated i.p. with 1×10⁷ pfu of VSV23 (blue), VSVST (pink), VSVXN2 (gold), VSVwt (aqua), or mock infected with diluent (grey). Uninfected animals were used as a control (red). Twenty days after immunization, splenocytes were harvested and cultured with syngeneic stimulator splenocytes that were either uninfected (FIG. 14A) or infected with VSVtsG41 (at the permissive temperature, 31° C.; FIG. 14B) at a ratio of 1:1. Triplicate cultures were incubated for 3 days at 37° C. 5% CO₂. T cell proliferation was then measured using the BrdU ELISA Assay Kit from Roche Applied Science. Data are presented as mean+/−standard deviation. All splenocytes cultured with VSVtsG41-infected stimulators showed similar levels of T cell proliferation, while those cultured with uninfected stimulators showed no proliferation above the background of mock-infected or uninfected control CD4 cells. The experiment shown is one of two replicate studies, with comparable results.

FIG. 15 shows that VSV23 elicits CTLs which recognize VSV-infected A20 cells. Cohorts of 6, 6 week old male BALB/cAnTac mice were immunized intraperitoneally with 1×10⁷ pfu of VSV23 (blue), VSVST (pink), VSVXN2 (gold), VSVwt (aqua), or mock infected with diluent (grey). Uninfected animals were used as a control (red). Twenty days later, splenocytes were harvested and cultured with syngeneic stimulator splenocytes either infected with VSVtsG41 or uninfected. After 5 days of incubation, effector cells were harvested, serially diluted and incubated with syngeneic A20 cells that were either infected with tsG41 or not infected. All splenocytes cultured with VSVtsG41 infected stimulators exhibited cytolytic activity against infected A20 cells, indicative of a memory response against VSV. There was no lysis of uninfected A20 cells, and virus infection of stimulator cells was required to induce CTL activity. This experiment was one of two replicate studies with comparable results.

FIG. 16 shows that neutralizing antibodies are present 20 days post infection in mice. Cohorts of 6, 6 week old male BALB/cAnTac mice were infected intranasally with 1×10³ pfu of VSV23 (blue), VSVST (pink), VSVXN2 (gold), or VSV wt (aqua). Uninfected animals were used as a control (grey). Blood samples, collected 20 days post infection from individuals, were serially diluted. 1×10³ pfu WT VSV was coincubated with the diluted serum for one hour. Triplicate samples were then used to infect monolayers of L929 cells; plaque assays were subsequently performed and used to determine antibody titer. All viral treatment groups showed similar levels of neutralizing antibodies to WT VSVs. This figure represents data from one experiment, mean+/−standard deviations are shown.

FIG. 17 shows NOS II expression in the olfactory bulb. 6 week old BALB/c mice were infected intranasally with VSV23, VSVST, VXN2, or VSVwt. Uninfected mice were used as a negative control. Brains were harvested on days 1, 3, 6, and 9. Sagittal sections were cut on a cryostat (20 μm) and stained with rabbit anti-mouse NOS II & donkey anti-rabbit Alexa Fluor® 546. VSV23 induces NOS H at day 1 post-infection.

FIG. 18 shows macrophage and microglia recruitment to the olfactory bulb: 6 week old BALB/c mice were infected intranasally with VSV23, VSVST, VSVXN2, or VSVwt. Uninfected mice were used as a negative control. Brains were harvested on days 1, 3, 6, and 9. Sagittal sections were cut on a cryostat (20 μm) and stained with rat anti-mouse CD11 b and goat anti-rat Alexa Fluor® 488. CD 11 b positive cells are detected in all infection groups and all times except VSV23 at day 9 post infection.

FIG. 19 shows neutrophil recruitment to the olfactory bulb. 6 week old BALB/c mice were infected intranasally with VSV23, VSVST, VSVXN2, or VSVwt. Uninfected mice were used as a negative control. Brains were harvested on days 1, 3, 6, and 9. Sagittal sections were cut on acryostat (20 μm) and stained with rat anti-mouse RB68C5 monoclonal antibody and goat anti-rat Alexa Fluor® 488. No difference in neutrophils recruitment was detected among the infection groups.

FIG. 20 shows CD4+& CD8+ recruitment to the olfactory bulb. 6 week old BALB/c mice were infected intranasally with VSV23, VSVST, VSVXN2, or VSVwt. Uninfected mice were used as a negative control. Brains were harvested on days 1, 3, 6, and 9. Sagittal sections were cut on a cryostat (20 mm) and stained with rat α-mouse L3T4, rat α-mouse Ly-2, and goat anti-rat Alexa Fluor® 488. No significant recruitment was seen in VSV23 infected animals. Control rVSVs and VSVwt induced CD4+ and CD8+T-cell responses.

FIG. 21 shows the infection with VSV23 Mitochondrial Dysfunction in JC cells. 1×10⁴ JC cells were plated in 96-well plates and incubated overnight at 37° C. Cells were then infected in triplicate at MOI=3 with VSV23 (blue), VSVST (pink), VSVXN2 (gold), VSVwt (aqua) or mock infected (grey) and incubated for 3, 6, 9, 12, 18, or 24 hours. The commercially available TACS MTT Cell Proliferation Assay Kit from R&D Systems was used per manufacturers instructions to measure mitochondrial dysfunction. Plates were read on an ELISA plate reader at 540 nm. All rVSVs are capable of inducing apoptosis in the JC cell line in vitro.

FIGS. 22A-D show that VSV23 induces CPE and cell death in multiple tumor lines in vitro. FIG. 22A uninfected JC cells; FIG. 22B VSV23 infected; FIGS. 22C and D: uninfected and VSV23-infected NB41A3. Images of cells at 8 hours post infection were acquired on an Olympus BH2-RFCA microscope (Olympus, Center Valley, Pa.) at 100× (FIGS. 22A, B) and 200× (FIGS. 22C, D). BHK21 cells were grown to 70% confluence in vitro in 10 cm tissue culture dishes and infected at MOI=0.1 with VSV23. Cells were incubated at 37° C. overnight and examined for evidence of apoptosis. Significant CPE was noted and 10 μl of virally infected supernatant was transferred to L929 adipocytes cells grown to 70% confluence in 10 cm tissue culture dishes. L929 cells were incubated overnight and CPE was detected. 10 μl of virally infected supernatant was transferred to NB41A3 neuroblastomas grown to 70% confluence in 10 cm tissue culture dishes. NB41A3 cells were incubated 8 hours, at which time initial signs of CPE were noted and photographed.

FIG. 23 shows that VSV23 infection inhibits tumor growth in vivo. Cohorts of N=4, 8-10 week old male BALB/c mice were injected subcutaneously on the left dorsal flank with 1×10⁷ JC cells suspended in 40 μl sterile HBSS. Ten days post-implantation, tumors were injected with 1×10⁷ pfu of VSV23 (blue solid), VSVST (pink horizontal stripes), VSVXN2 (gold vertical stripes) diluted in 40 μl of PBS or vehicle alone (red spotted). Viral treatments were repeated on days 3 and 5 after the initial treatment. VSV23 treated tumors decrease in size during the first six days of monitoring after treatment. The average size of VSV23 treated tumors increases 8 days after treatment. Tumors treated with control viruses were of decreased size compared to untreated tumors through the first 5 days of monitoring. By the end of the 14 day monitoring period control virus treated tumors were of similar size to untreated tumors, while VSV23 infected tumors remained significantly smaller than untreated tumors (p<0.005). Data shown are representative of three replicate experiments and error bars represent standard deviation.

FIGS. 24A-P show that inflammatory cells infiltrate rVSV treated tumors. CD8⁺T cells (FIGS. 24A, E, I, and M), CD4⁺T cells (FIGS. 24B, F, J, and N), macrophages (FIGS. 24C, G, K, and O), and neutrophils (FIGS. 24C, G, K, and O). Cohorts of N=4, 8-10 week old male BALB/c mice were injected subcutaneously on the left dorsal flank with 1×10⁷JC cells. Ten days post-implantation, tumors were injected with 1×10⁷ pfu of VSV23 (FIGS. 24A-D), VSVST (FIGS. 24E-H), VSVXN2 (FIGS. 24I-L), or vehicle alone (FIGS. 24M-P). Viral treatments were repeated on days 3 and 5 after the initial treatment. Fourteen days after treatment was initiated, tumors were harvested, frozen, sliced into 18 μm thick sections, and treated with antibodies specific for cell types and secondary antibodies as described in Table 4; tissues were counterstained with DAPI to label nuclei. Images were obtained using a Leica SP5 confocal microscope at 400× magnification.

FIG. 25 shows that VSV23 treatment results in enhanced memory CTL responses against JC tumor cells in vitro. Cohorts of N=4, 8-10 week old male BALB/c mice were injected subcutaneously on the left dorsal flank with 1×10⁷ JC cells. Ten days post-implantation, tumors were injected with 1×10⁷ pfu of VSV23 (blue), VSVST (pink), or VSVXN2 (gold) or vehicle alone (red). Viral treatments were repeated on days 3 and 5 after the initial treatment. 14 days after treatment was initiated, splenocytes were harvested and cultured with JC cells in vitro. Cultured naïve T cells from non-tumor bearing animals were used as a negative control. Splenocytes from all tumor bearing animals exhibited T cell responses against tumor cells; however splenocytes from VSV23 treated animals exhibited enhanced JC tumor killing capacity. Data presented are means±standard deviation and are representative of three replicate experiments.

### DETAILED DESCRIPTION OF THE INVENTION

One aspect of the present invention is directed to a modified recombinant replicable vesiculovirus comprising vesiculovirus N, P, L proteins, and a replicable vesiculovirus genomic sense (−) RNA comprising a nucleic acid molecule encoding for IL23.

In a preferred embodiment, the modified recombinant replicable vesiculovirus comprising vesiculovirus N, P, L proteins, and a replicable vesiculovirus genomic sense (−) RNA comprises a nucleic acid molecule that encodes for the p40 and p19 subunits of the IL23 protein. The two subunit could be preferably linked together with a spacer peptide.

In one embodiment, the recombinant vesiculovirus has an IL23 encoding nucleic acid molecule present in the vesiculovirus genomic sense (−) RNA as an insertion or as a replacement. The RNA complementary to the nucleic acid molecule which encodes for IL23 protein is either inserted into a nonessential portion of the replicable vesiculovirus genomic sense (−) RNA, or replaces a nonessential portion of the genomic sense (−) RNA.

In a preferred embodiment, the vesiculovirus is vesicular stomatitis virus. Many vesiculoviruses known in the art can be made recombinant according to the present invention. Examples of such vesiculoviruses are listed in Table 1.

TABLE 1
Members of the vesiculovirus genus

| Virus | Source of virus in nature |
|---|---|
| VSV-New Jersey | Mammals, mosquitoes, midges, |
| | blackflies, houseflies |
| VSV-Indiana | Mammals, mosquitoes, sandflies |
| Alagoas | Mammals, sandflies |
| Cocal | Mammals, mosquitoes, mites |
| Jurona | Mosquitoes |
| Carajas | Sandflies |
| Maraba | Sandflies |
| Piry | Mammals |
| Calchaqui | Mosquitoes |
| Yug | Bogdanovac Sandflies |
| Isfahan | Sandflies, ticks |
| Chandipura | Mammals, sandflies |
| Perinct | Mosquitoes, sandflies |
| Porton-S | Mosquitoes |

One aspect of the present invention is directed to a host cell comprising (i.e., transformed, transfected or infected with) the modified recombinant vesiculovirus or vectors described herein. The host cell also further comprises a first recombinant nucleic acid molecule that can be transcribed to produce an RNA comprising a vesiculovirus antigenomic (+) RNA containing the vesiculovirus promoter for replication, in which a region of the RNA nonessential for replication of the vesiculovirus has been inserted into or replaced by the IL23 encoding RNA. The host cell also comprises a second recombinant nucleic acid molecule encoding a vesiculovirus N protein, a third recombinant nucleic acid molecule encoding a vesiculovirus L protein, and a fourth recombinant nucleic acid molecule encoding a vesiculovirus P protein.

In another embodiment the host cell comprises first, second, third, and fourth plasmid vectors. The first DNA plasmid vector comprises the following operatively linked components: (i) a bacteriophage RNA polymerase promoter; (ii) a first DNA molecule that is transcribed in the cell to produce an RNA comprising (A) a vesiculovirus antigenomic (+) RNA containing the vesiculovirus promoter for replication, in which a region of the RNA nonessential for replication of the vesiculovirus has been inserted into or replaced by the IL23 encoding RNA, and (B) a ribozyme immediately downstream of said antigenomic (+) RNA, that cleaves at the 3′ terminus of the antigenomic RNA; and (iii) a transcription termination signal for the RNA polymerase. The second DNA plasmid vector comprises the following operatively linked components: (i) the bacteriophage RNA polymerase promoter; (ii) a second DNA encoding a N protein of the vesiculovirus; and (iii) a second transcription termination signal for the RNA polymerase. The third DNA plasmid vector comprises the following operatively linked components: (i) the bacteriophage RNA polymerase promoter; (ii) a third DNA encoding a P protein of the vesiculovirus; and (iii) a third transcription termination signal for the RNA polymerase. The fourth DNA plasmid vector comprising the following operatively linked components: (i) the bacteriophage RNA polymerase promoter; (ii) a fourth DNA encoding a L protein of the vesiculovirus; and (iii) a fourth transcription termination signal for the RNA polymerase. The host cell also includes a recombinant vaccinia virus comprising a nucleic acid molecule encoding the bacteriophage RNA polymerase. In the cell, the first DNA is transcribed to produce said RNA, the N, P, and L proteins and the bacteriophage RNA polymerase are expressed, and the modified recombinant replicable vesiculovirus is produced that has a genome that is the complement of said antigenomic RNA.

The recombinant vesiculoviruses of the present invention may be produced with an appropriate host cell containing vesiculovirus cDNA. The cDNA comprises a nucleotide sequence encoding a heterologous target molecule which could be a protein or a combination of proteins. In addition to IL23, such proteins can be, for example, cytokines, a protein/peptide that mediates a disease or disorder which is readily known in the art such as p52 gene in Plasmodium falciparum, as well as epitopes (antigenic determinants) from various parasites and bacteria such as Eimeria spp, Vibrio cholerae, Streptococcus pneumoniae. The nucleic acid encoding a heterologous protein can be inserted in a region non-essential for replication, or a region essential for replication, in which case the VSV is grown in the presence of an appropriate helper cell line. In some examples, the production of recombinant VSV vector is in vitro using cultured cells permissive for growth of the VSV.

Primary cells lacking a functional IFN system, or in other examples, immortalized or tumor cell lines can be used as host cells. A vast number of cell lines commonly known in the art are available for use. Both prokaryotic and eukaryotic host cells, including insect cells, can be used as long as sequences requisite for maintenance in that host, such as appropriate replication origin(s), are present. For convenience, selectable markers are also provided. Suitable prokaryotic host cells include bacterial cells, for example, E. coli, B. subtilis, and mycobacteria. Useful eukaryotic host cells include yeast, insect, avian, plant, C. elegans (or nematode), and mammalian host cells. Examples of fungi (including yeast) host cells are S. cerevisiae, species of Candida, including C. albicans and C. glabrata, Aspergillus nidulans, Schizosaccharomyces pombe (S. pombe), and Pichia pastoris. Examples of mammalian cells are COS cells, baby hamster kidney cells (BHK-21), mouse L cells (L929), LNCaP cells, Chinese hamster ovary (CHO) cells, human embryonic kidney (HEK) cells, and African green monkey cells. Xenopus laevis oocytes or other cells of amphibian origin may also be used. These and other useful cell lines are publicly available for example, from the ATCC and other culture depositories.

In carrying out the present invention, an isolated nucleic acid molecule which encodes for the recombinant vesiculovirus and has an IL23 encoding nucleic acid molecule either inserted in or replacing a nonessential portion of the vesiculovirus genomic sense (−) RNA. The recombinant production of viral vectors, viral particles, and other proteins encoded by nucleic acid molecules are well known in the art. A detailed description of suitable techniques and components for the recombinant production of vesiculoviruses related to that of the present invention are described in detail in U.S. Pat. No. 7,153,510 to Rose et al., which is hereby incorporated by reference in its entirety. In particular, this reference includes a lengthy description of components like promoters, termination sequences, ribozyme sequences, antigens, expression vectors, and host cells. Also, taught by U.S. Pat. No. 7,153,510 to Rose, et al. are techniques relevant to recombinant production of vesiculoviruses including combining nucleic acid molecules (e.g., restriction sites, intergenic regions, and cleaving and ligating techniques), mutagenesis, transformation and transaction, culturing, and purification. These and other aspects of the present invention are more fully described in U.S. Pat. No. 7,153,510 to Rose, et al., which is hereby incorporated by reference in its entirety.

Another aspect of the present invention is directed to a recombinant, replicating and infectious vesicular stomatitis virus (VSV) particle which comprises a functional RNA dependent RNA polymerase (L), a vesiculovirus phosphoprotein (P), a vesiculovirus nucleocapsid (N), vesiculovirus protein selected from the group consisting of glycoprotein (G) and matrix (M), a 3′ non-coding RNA sequence, and a 3′ to 5′ RNA coding sequence, which encodes the vesiculovirus L, P, N and a vesiculovirus protein required for assembly of budded infectious particles and including a nucleic acid molecule which encodes for IL23, wherein the nucleic acid molecule encoding IL23 is inserted at an intergenic junction, a 5′ non-coding RNA sequence, wherein all components are from the same type of VSV.

In a preferred embodiment, the recombinant, replicating and infectious vesicular stomatitis virus (VSV) particle comprises the nucleic acid molecule which encodes for a single chain protein composed of the p40 and p19 subunits of the IL23 protein.

Another aspect of the present invention is directed to a method of treating cancer in a subject. This method involves selecting a subject with cancer and administering to the subject the recombinant replicable vesiculovirus modified with IL23 under conditions effective to treat cancer.

VSV preferentially replicates in malignant cells eventually leading to apoptosis or oncolysis. This selective replication of VSV in malignant or tumor cells is in part due to defective interferon (IFN) system. Normal cells have a functional IFN system and are therefore protected from the VSV virus (Balachandran et al., “Defective Translational Control Facilitates Vesicular Stomatitis Virus Oncolysis,” Cancer Cell 5:51-65 (2004); Barber, “Vesicular Stomatitis Virus as an Oncolytic Vector,” Viral Immunol. 17(4):516-27 (2004); Stodjl et al., “Exploiting Tumor-specific Defects in the Interferon Pathway with a Previously Unknown Oncolytic Virus,” Nature Medicine 6:821-825 (2000), which are hereby incorporated by reference in their entirety). This preferential targeting of cancerous cells over normal cells makes VSV an attractive therapeutic candidate for use in treating cancer.

The present invention provides methods for producing oncolytic activity in a tumor cell and/or malignant cell and/or cancerous cell by contacting the cell, including, for example, a tumor cell or a malignant cell in metastatic disease, with a recombinant vesiculovirus or vesiculovirus vector modified with IL23 protein of the present invention. The vesiculovirus or vesiculovirus vector exhibits oncolytic activity against the cell.

The use of vesicular stomatitis virus (VSV) as an oncolytic agent has several advantages over other virus delivery systems presently used in tumor therapy such as adenoviruses and retroviruses. Foremost, VSV has no known transforming abilities. The envelope glycoprotein (G) of VSV is highly tropic for a number of cell-types and should be effective at targeting a variety of tissues in vivo. VSV appears to be able to replicate in a wide variety of tumorigenic cells and not, for example, only in cells defective in selective tumor suppressor genes such as p53. VSV is able to potently exert its oncolytic activity in tumors harboring defects in the Ras, Myc, and p53 pathways, cellular aberrations that occur in over 90% of all tumors.

The vesiculovirus may be used in conjunction with other treatment modalities for producing oncolytic activity, or tumor suppression, including but not limited to chemotherapeutic agents known in the art, radiation and/or antibodies. The present invention can also be carried out with a VSV vector or viral particle that encodes for a cancer specific antigen which can elicit an immune response against the cancerous cell.

Cancers treatable in accordance with the present invention include melanoma, breast cancer, prostate cancer, cervical cancer, hematological-associated cancer, a solid tumor, or a cancer caused due to a defect in the tumor suppressor pathway. VSV in accordance with the present invention is useful in inducing cell death in transformed human cell lines including those derived from breast (MCF7), prostate (PC-3), or cervical tumors (HeLa), as well as a variety of cells derived from hematological-associated malignancies (HL 60, K562, Jurkat, BC-1). BC-1 is positive for human herpesvirus-8 (HHV-8), overexpresses Bcl-2 and is largely resistant to a wide variety of apoptotic stimuli and chemotherapeutic strategies. VSV would be expected to induce apoptosis of cells specifically transformed with either Myc or activated Ras and transformed cells carrying Myc or activated Ras or lacking p53 or over expressing Bcl-2. It has been shown that several human cancer cell lines are permissive to VSV replication and lysis. Therefore administration of a VSV vector or viral particle of the present invention or a composition comprising such a vector or particle would produce oncolytic activity in a variety of malignant cells or tumor cells.

The present invention encompasses treatment using a vesiculoviruses or vector(s) in individuals (e.g., mammals, particularly humans) with malignant cells and/or tumor cells susceptible to vesiculovirus infection, as described above. Also indicated are individuals who are considered to be at risk for developing tumor or malignant cells, such as those who have had previous disease comprising malignant cells or tumor cells or those who have had a family history of such tumor cells or malignant cells. Determination of suitability of administering VSV vector(s) of the invention will depend on assessable clinical parameters such as serological indications and histological examination of cell, tissue or tumor biopsies. Generally, a composition comprising the virus(es) or vector(s) of the present invention in a pharmaceutically acceptable excipient(s) is administered.

In another aspect, the present invention relates to a method for treating or preventing a disease or disorder mediated by a peptide or protein. This method involves selecting a subject in need of treatment or prevention of the disease or disorder. The IL23 modified recombinant vesiculovirus or vector. The viruses or vectors also encode for the protein or peptide which mediate the disease or disorder. The leads to induction of an immune response against the pathogenic peptide or protein.

A vaccine can be formulated in which the immunogen is one or several modified recombinant vesiculovirus(es), in which a foreign RNA in the genome directs the production of an antigen in a host to elicit an immune (humoral and/or cell mediated) response in the host that is prophylactic or therapeutic. The foreign RNA contained within the genome of the recombinant vesiculovirus, upon expression in an appropriate host cell, produces a protein or peptide that is antigenic or immunogenic. The replicable IL23 modified vesiculovirus genomic sense (−) RNA is further modified by insertion of an RNA complementary to a nucleic acid molecule which encodes for a peptide or protein in a nonessential portion of the vesiculovirus genomic sense (−) RNA, or by replacement of a nonessential portion of the replicable vesiculovirus genomic sense (−) RNA by an RNA complementary to the nucleic acid molecule which encodes for a peptide or protein. The peptide or protein displays the antigenicity or immunogenicity of an epitope (antigenic determinant) of a pathogen and the administration of the vaccine is carried out to prevent or treat an infection by the pathogen and/or the resultant infectious disorder or disease and/or other undesirable correlates of infection. The peptide or protein can be the immunogenic portion of an antigen of a pathogenic organism, wherein the pathogenic organism belongs to the group consisting of bacteria, virus, fungi, parasites, non-human pathogens, and human pathogens.

In a preferred embodiment, the antigen is a cancer related or tumor related antigen. The administration of the vaccine is carried out to prevent or treat tumors (particularly, cancer).

The vaccines of the invention may be multivalent or univalent. Multivalent vaccines are made from recombinant viruses that direct the expression of more than one antigen, from the same or different recombinant viruses. The virus vaccine formulations of the invention comprise an effective immunizing amount of one or more recombinant vesiculoviruses (live or inactivated, as the case may be) and a pharmaceutically acceptable carrier or excipient. Subunit vaccines comprise an effective immunizing amount of one or more antigens and a pharmaceutically acceptable carrier or excipient.

The recombinant vesiculoviruses that express an antigen can also be used to recombinantly produce the antigen in infected cells in vitro, to provide a source of antigen for use in for example immunoassays, and thus to diagnose infection or the presence of a tumor and/or monitor immune response of the subject subsequent to vaccination.

The antibodies generated against the antigen by immunization with the recombinant viruses of the present invention also have potential uses in passive immunotherapy and generation of antiidiotypic antibodies.

The vaccine formulations of the present invention can also be used to produce antibodies for use in passive immunotherapy, in which short-term protection of a host is achieved by the administration of pre-formed antibody directed against a heterologous organism.

The antibodies generated by the vaccine formulations of the present invention can also be used in the production of antiidiotypic antibody. The antiidiotypic antibody can then in turn be used for immunization, in order to produce a subpopulation of antibodies that bind the initial antigen of the pathogenic microorganism (Jerne, “Towards a Network Theory of the Immune System,” Ann. Immunol. (Paris) 125c:373-89 (1974); Jerne et al., “Recurrent Idiotypes and Internal Images,” EMBO J. 1:234-7 (1982), which are hereby incorporated by reference in their entirety).

Another aspect of the present invention is related to a composition containing the VSV vectors or viral particles of the present invention as described supra and a pharmaceutically acceptable carrier or other pharmaceutically acceptable components.

As will be apparent to one of ordinary skill in the art, administering any of the vectors or viral particles of the present invention may be carried out using generally known methods. Typically, the agents of the present invention can be administered orally, parenterally, for example, subcutaneously, intravenously, intramuscularly, intraperitoneally, by intranasal instillation, by application to mucous membranes, such as, that of the nose, throat, and bronchial tubes or by direct contact to the cancer cells, by direct injection into the cancer cells or by intratumoral injection. The viral particles or the vectors can also be contained in a cell line infected with the virus and administered by many methods including but not limited to, intratumoral, intravenous, intraperitoneally, or subcutaneously. They may be administered alone or with suitable pharmaceutical carriers, and can be in solid or liquid form such as, tablets, capsules, powders, solutions, suspensions, or emulsions. The amount of vector(s) to be administered will depend on several factors, such as route of administration, the condition of the individual, the degree of aggressiveness of the malignancy, and the particular vector employed. Effective doses of the vector or viral particle of the present invention may also be extrapolated from dose-response curves derived from animal model test systems. Also, the vector may be used in conjunction with other treatment modalities. Formulations also include lyophilized and/or reconstituted forms of the vectors (including those packaged as a virus) of the present invention.

The virus vaccine formulations of the present invention comprise an effective immunizing amount of one or more recombinant vesiculoviruses (live or inactivated, as the case may be) and a pharmaceutically acceptable carrier or excipient. Subunit vaccines comprise an effective immunizing amount of one or more antigens and a pharmaceutically acceptable carrier or excipient. Pharmaceutically acceptable carriers are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The vectors or viral particles of the present invention may be orally administered, for example, with an inert diluent, with an assimilable edible carrier, enclosed in hard or soft shell capsules, compressed into tablets, or incorporated directly with the food of the diet. Such compositions and preparations should contain at least 0.1% of active compound. The percentage of the vectors or viral particles in these compositions may, of course, be varied and may conveniently be between about 2% to about 60% of the weight of the unit. The amount of active agent in such therapeutically useful compositions is such that a suitable dosage will be obtained. Preferred compositions according to the present invention are prepared so that an oral dosage unit contains between about 1 and 250 mg of active compound.

Pharmaceutically acceptable carriers for oral administration are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration. The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both.

These vectors or viral particles may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms. Formulations for parenteral and nonparenteral drug delivery are known in the art and are set forth in Remington's Pharmaceutical Sciences, 19th Edition, Mack Publishing (1995), which is hereby incorporated by reference in its entirety.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents of the present invention may also be administered directly to the airways in the form of an aerosol. For use as aerosols, the agents of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Suitable subjects to be treated in accordance with the present invention are subjects that are at risk of developing or have developed cancer or are in need of vaccination against disease/s. Such subjects include human and non-human animals, preferably mammals or avian species. Exemplary mammalian subjects include, without limitation, humans, non-human primates, dogs, cats, rodents, cattle, horses, sheep, and pigs. Exemplary avian subjects include, without limitation chicken, quail, turkey, duck or goose. In the use of vectors or viral particles of the present invention, the subject can be any animal in which the vector or virus is capable of growing or replicating.

The present invention is illustrated, but not limited, by the following examples.

### EXAMPLES

### Example 1

### Materials and Methods

### Cells Lines and Viruses

A20 (syngeneic H-2^{d} MHC I and MHC II-expressing), BHK-21 baby hamster kidney cells, JC murine mammary gland adenocarcinoma-derived cells, L929 murine adipocytes, NB41A3 murine neuroblastoma cells, Raw 264.7 murine macrophage derived cells, and Yac-1 were all purchased from the American Type Culture Collection (Manassas, Va.). BHK-21 cells were grown in Minimum Essential Media (MEM) (Mediatech, Manassas, Va.) with 1% non-essential amino acids (NEAA), 1% penicillin-streptomycin (pen-strep) and 10% fetal bovine serum (FBS), A20, JC, and YAC-1 cells grown in RPMI1640 (Mediatech, Manassas, Va.) with 1% pen-strep and 10% FBS, L929 cells grown Dulbecco's Modification of Eagles Medium (DMEM) (Mediatech, Manassas, Va.) with 1% pen-strep, 1% HEPES buffer, 1% L-glutamine and 10% FBS, NB41A3 grown in F-12K media (Mediatech, Manassas, Va.) with 2.5 FBS and 15% horse serum, and Raw 264.7 cells grown in DMEM (Mediatech, Manassas, Va.) with 1% pen-strep and 10% FBS. VSV Indiana strain, San Juan serotype, was originally obtained from Alice S. Huang (then at The Children's Hospital; Boston, Mass.). VSV tsG41 was obtained from Alice S. Huang and has been used for in vitro immunological studies (Reiss et al., “VSV G Protein Induces Murine Cytolytic T Lymphocytes,” Microb. Pathog. 1(3):261-7 (1986), which is hereby incorporated by reference in its entirety).

### Viral Plaque Assays

Monolayers of mouse L929 cells were prepared in 24-well plates (Becton Dickinson; Franklin Lakes, N.J.) at least seven hours prior to infection. Ten-fold serial dilutions of viral supernatants were prepared in serum free DMEM and added to aspirated L929 monolayers. After 30 minutes, unadsorbed virus was removed via aspiration and 1 ml melted 0.9% Bacto-agar in 1× Joklick medium (MEM+125 mM NaHCO₃+10% FBS+2% glutamine+1% nonessential amino acids+1% penicillin/streptomycin) was added to each well. Following incubation at 37° C. and 5% CO₂ for 22 hours, each well was overlaid with 0.5 ml 10% formalin on the agar plugs and fixed for 20-30 minutes at room temperature (RT). Subsequently, each agar plug was carefully removed, to avoid scrapping the cell monolayer, and enough 0.5% cresyl violet was added to each well to cover and stain the fixed cells. Finally, after three minutes incubation at RT, the cresyl violet was washed away with water, dried and plaques counted.

### One-Step Growth Curve

L929 cells were grown to 90% confluence in 24-well plates and infected with VSV23, VSVST, VSVXN2 or wild-type VSV (VSVwt) at a MOI=1 for 30 minutes at RT. Wells were washed with Hank's Balanced Salt Solution (HBSS) to remove unadsorbed virus and media was added to each well. Aliquots of media were removed at 1.5, 3, 6, 12 and 24 hours and stored at −80° C. Viral titers were determined by plaque assay on L929 cells. All samples were assayed in triplicate and the experiment repeated twice.

### ELISA for Virally Produced IL-23

BHK-21 cells were infected with VSV23, VSVST, VSVXN2 or VSVwt at MOI=0.1 and incubated overnight at 37° C. and 5% CO₂. Uninfected BHK-21 cells were used as a negative control. Supernatants were harvested and subjected to ELISA analysis specific for the p40 subunit of IL-23 using the Mouse IL-12/IL-23 Total p40 ELISA kit (eBioscience, San Diego, Calif.).

### In Vivo Studies

All procedures involving animals were approved by and performed according to the guidelines of The University Animal Welfare Committee of New York University. Six-week or eight to ten-week old male BALB/cAnNTac (BALB/c) mice were purchased from Taconic Farms, Inc. (Germantown, N.Y.), housed under standard conditions and fed ad libitum. Mice were housed for one week prior to initiation of experiments.

vIL23 RT-PCR Bioactivity Assay

Raw 264.7 cells were added to 6-well plates from Fisher Scientific (Pittsburgh, Pa.) with DMEM supplemented with 10% heat inactivated FBS and 1% pen/strep. Spleens were aseptically harvested from eight-week old male BALB/c mice and teased into single cell suspensions. CD4⁺T cells were isolated using the Dynal® Mouse CD4 Negative Isolation Kit (Oslo, Norway) and added to 6-well plates from Fisher Scientific (Pittsburgh, Pa.) with DMEM supplemented with 10% heat inactivated FBS and 1% pen/strep. Raw 264.7 and CD4⁴ T cells were treated with ultraviolet-inactivated supernatants from BHK-21 cells containing 500 pg of VSV23 induced IL-23 (vIL-23). UV-inactivated supernatants containing 500 pg of virally induced IL-12 (vIL-12) from BHK-21 cells infected with a rVSV expressing IL-12, a gift of Dr. Savio Woo (Mt. Sinai School of Medicine, NY, N.Y.) were also tested. 500 pg of recombinant IL-23 (rIL-23) or rIL-12 (R&D Systems Minneapolis, Minn.) were used to treat cells as positive cytokine controls and supernatant from untreated/uninfected BHK-21 cells was used as a negative control. Samples were incubated at 37° C. and 5% CO₂ for 6 hours and RNA was isolated with Trizol® reagent (Invitrogen, San Diego, Calif.). RNA was subjected to reverse transcriptase PCR (rtPCR) for detection IFN-γ or TN F-α mRNA. β-actin was used as a housekeeping control for the reaction. Primers are listed in Table 2.

TABLE 2
Primers for IL-23 Induced Cytokine mRNA

| Primer | | |
|---|---|---|
| Designation | Sequence (5′ to 3′) | |
| JM005 IFNγ | gctttgcagctcttcctcat | (SEQ ID NO: 8) |
| JM006 IFNγ | tgagctcattgaatgcttgg | (SEQ ID NO: 9) |
| JM017 TNFα | gaactggcagaagaggcact | (SEQ ID NO: 10) |
| JM01 8 TNFα | cggactccgcaaagtctaag | (SEQ ID NO: 11) |
| JM01 9 β-Actin | aagagctatgagctgcctga | (SEQ ID NO: 12) |
| JM020 β-Actin | tacggatgtcaacgtcacac | (SEQ ID NO: 13) |

### Natural Killer Cell Activity Assay

Cohorts of six-week old male BALB/c mice were inoculated i.p. (intraperitonial) with 1×10⁷ plaque forming units (pfu) of VSV23, VSVST, VSVXN2, VSVwt, or mock infected as a control. Three days later, spleens from individual mice were harvested, teased into a single cell suspension, and resuspended in MEM, 10% FBS, 1% Pen-Strep. 1×10⁴ YAC-1 cells were plated in 96-well V-bottom plate wells in 100 μl of MEM supplemented with 10% FBS and 1% Pen-Strep. Splenocytes were co-incubated with YAC-1 cells in triplicate at ratios of 200:1, 100:1, 50:1, 25:1, and 12.5:1, and 6.25:1 in a total volume of 100 μl. Plates were centrifuged at 200×g for five minutes to improve contact between cells and incubated for four hours at 37° C., 5% CO₂. The CytoTox 96™ non-radioactive cytotoxicity kit (Promega, Madison, Wis.) was used per manufacturer's instructions to determine NK mediated cytolytic activity. Results were read on a Biorad 550 series microplate reader (Hercules, Calif.) at 490 nm. Results are representative of two replicate experiments.

### Cytolytic T Cell Activity Assay

Cohorts of N=6, six-week old male BALB/c mice were injected i.p. with 1×10⁷ pfu of VSV23, VSVST, VSVXN2, or VSVwt, to produce responder cells. Mock-injected mice were used as a negative control and as a source of naive controls for effector cells. Twenty days after immunization, spleens from individual mice were harvested, teased into a single cell suspension, and resuspended in MEM, 10% FBS, 1% Pen-Strep. Stimulator cells were prepared by infecting naive splenocytes with VSV tsG41 at MOI=5 at the permissive temperature of 33° C. for 1 hour (Browning et al., “Replication-Defective Viruses Modulate Immune Responses,” J. Immunol. 147(8):2685-91 (1991), which is hereby incorporated by reference in its entirety). Cells were washed in HBSS to remove unadsorbed virions. 5×10⁶ responder cells were cultured with 1×10⁶ VSV tsG41 stimulator cells or with uninfected stimulator cells for 5 days in DMEM, 10% FBS, 1% Pen-Strep, 5 mM 2-mercaptoethanol, and 1% L-glutamine at 37° C., 5% CO₂. A20 cells (syngeneic H-2^{d} MHC I and MHC II-expressing) were used as target cells (Browning et al., “Cytolytic T Lymphocytes From the BALB/c-H-2dm2 Mouse Recognize the Vesicular Stomatitis Virus Glycoprotein and are Restricted by Class II MHC Antigens,” J. Immunol. 145(3):985-94 (1990); Reiss et al., “VSV G Protein Induces Murine Cytolytic T Lymphocytes,” Microb. Pathog. 1(3):261-7 (1986), which are hereby incorporated by reference in their entirety). Target cells were either infected with VSVwt at M01=3 or mock-infected and plated in 96 well V-bottom plates at 1×10⁴ cells per well. Responder cells from individual mice of each treatment group were added to target cells in triplicate at effector to target ratios of 100:1, 50:1, 25:1, and 12:1. Plates were centrifuged for five minutes at 200×g to improve cell contacts and incubated for four hours at 37° C., 5% CO₂. The CytoTox 96™ non-radioactive cytotoxicity kit (Promega, Madison, Wis.) was used per manufacturer's instructions to determine T cell mediated cytolytic activity. Results were read on a Biorad 550 series microplate reader at 490 nm. Results are representative of two replicate experiments.

### Memory T Cell Proliferation Assay

Cohorts of N=6, six-week-old male BALB/c mice were inoculated i.p. with 1×10⁷ pfu of VSV23, VSVST, VSVXN2, or VSVwt to produce responder cells. Mock-treated mice were used as a negative control. Twenty days later, spleens were harvested, teased into a single cell suspension, and resuspended in MEM supplemented with 10% FBS and 1% Pen-Strep. Stimulator cells were prepared by treating naive spleen cells with five pfu of VSVtsG41 per cell at the permissive temperature of 33° C. for 1 hour. Cells were washed in HBSS to remove unabsorbed virions. 1×10⁵ responder cells from individual mice were seeded in a 96-well plate, in triplicate, with either 1×10⁵ of VSVtsG41 infected or uninfected stimulator cells and allowed to incubate for three days in MEM supplemented with 10% FBS, 1% Pen-Strep, 2-mercaptoethanol, and L-glutamine at 37° C., 5% CO₂. The Cell Proliferation ELISA, BrdU (colorimetric) kit (Roche Diagnostics, Indianapolis, Ind.) was utilized per manufacturer's instructions and the results read on a Biorad 550 series microplate reader at 490 nm. Results are representative of two replicate experiments.

### Neutralizing Antibody Titer Assay

Cohorts of six-week old male BALB/c mice were infected La (intranasal) with 1×10³ pfu of VSV23, VSVST, VSVXN2, or VSVwt. Uninfected animals were used as a control. Blood samples were collected from the ocular plexus, 20 days pi (post infection) from surviving individual animals and allowed to clot overnight at 4° C. Serum was diluted in phosphate buffered saline (PBS) in serial five-fold steps. 1×10³ pfu of VSVwt was added to each dilution and incubated at 37° C. 5% CO₂ for one hour. Plaque assays were performed in triplicate samples on L929 cells and neutralizing antibody titers were calculated. Results are representative of three replicate experiments.

Plaque Assay for Viral Titers in the CNS after Intranasal VSV Infection

Cohorts of N=6, six week-old male BALB/c mice were infected La (intranasal) with 1×10³ pfu or 1×10⁶ of VSV23, VSVST, or VSVXN2. Individuals were sacrificed on days 1, 3, 6, and 9 post-infection and brains were divided sagittally. One half was reserved for immunohistochemical staining. The other brain half was individually homogenized, and an aliquot was serially diluted, and assayed in triplicate by plaque assay on L929 cells for the presence of VSV. Geometric mean titers were calculated for each cohort.

Greiss Assay for Nitric Oxide Levels in the CNS after Intranasal VSV Infection

Cohorts of N-6, six week-old male BALB/c mice were infected with 1×10³ pfu or 1×10⁶ of VSV23, VSVST, or VSVXN2. Individuals were sacrificed on days 1, 3, 6, and 9 post-infection and brains were divided sagittally. One half was reserved for immunohistochemical staining. The other brain half was individually homogenized. Tissue homogenate samples for NO assays were pre-cleared of solid material by centrifugation. The Total Nitric Oxide Assay Kit (Pierce, Rockford, Ill.) was used as per manufacturer's instructions to convert nitrate to nitrite from individual samples. Equal volumes of experimental sample and Greiss reagent (1% sulfanilamide, 0.1% n-1-naphthylethylene-diamine, and 5% H₃PO₄; Sigma-Aldrich, St. Louis, Mo.) were incubated at RT for 15 minutes and results were read on a Biorad 550 series microplate reader at 540 nM.

Immunohistochemical Staining and Microscopy of rVSV Infected Brains

Staining was performed in order to detect viral antigens. Brain hemispheres were frozen in Tissue-Tek OCT compound (Sakura, Torrance, Calif.), sliced into 18 μm thick sections using the Leica CM1850UV Cryostat (Leica, Bannockburn, Ill.) and placed on poly-L-lysine-coated slides. Sections were fixed in 5% paraformaldehyde for 10 minutes. The sections were then washed twice with PBS and incubated in 20 μg/ml goat anti-mouse IgG (Jackson Immunoresearch Laboratories Inc., West Grove, Pa.) for 45 minutes when necessary. Sections were then incubated in PBS with Blotto for 45 minutes. The slides were once again washed with PBS and incubated overnight in primary antibodies. Slides were then washed with PBS and incubated in secondary antibody for 45 minutes. After incubation with secondary antibody, the sections were washed with PBS and mounted with Vectorshield Mounting Medium (Vector Laboratories, Burlingame, Calif.). Primary antibodies (Abeam, Cambridge Mass.) and secondary antibodies (Invitrogen, Carlsbad, Calif.) used are shown in Table 3. Slides were covered with number 1.5 cover slips (Fisher; Waltham, Mass.), and viewed using a Leica SP5 confocal microscope at 400× magnification or on an Olympus BH2-RFCA microscope (Olympus, Center Valley, Pa.).

TABLE 3
Primary and Secondary Antibodies

| Primary | | | | |
|---|---|---|---|---|
| mAb | Specificity | Dilution | Secondary Ab | Dilution |
| rat α-mouse | Macrophages | 1:200 | goat α-rat | 1:100 in PBS |
| CD11b | | in PBS | Alexa Fluor ® | & Blotto |
| | | | 488 | |
| rat α-mouse | Neutrophils | 1:200 | goat α-rat | 1:100 in PBS |
| GR-1 | | in PBS | Alexa Fluor ® | & Blotto |
| | | | 488 | |
| rat α-mouse | CD4 T cells | 1:200 | goat α-rat | 1:100 in PBS |
| L3T4 | | in PBS | Alexa Fluor ® | & Blotto |
| | | | 488 | |
| rat α-mouse | CD8 cells | 1:200 | goat α-rat | 1:100 in PBS |
| LyT-2 | | in PBS | Alexa Fluor ® | & Blotto |
| | | | 488 | |
| rabbit α- | iNOS in | 1:100 | donkey α- | 1:100 in PBS |
| mouse NOS II | Macrophages/ | in PBS | rabbit Alexa | & Blotto |
| | Microglia | | Fluor ® 633 | |
| sheep | VSV proteins | 1:200 | rabbit α- | 1:100 in PBS |
| polyclonal α- | | in PBS | sheep Alexa | & Blotto |
| VSV | | | Fluor ® 488 | |

### Intranasal Infection Morbidity and Mortality Assay

Cohorts of N=10, six-week old male BALB/c mice were infected in with 1×10⁶ pfu of VSV23, VSVST, or VSVXN2 and monitored daily for 15 days. Mice were weighed daily. Hind-limb paralysis or weight loss that exceeded 30% of starting weight were considered end points for the experiment; mice were humanely sacrificed if they were found to have weight-loss or paralysis. Mice were individually scored blind on a subjective six point scale (0-5): “0” for no symptoms, “1” for lack of grooming behavior, “2” for hunched and severely lethargic mice, “3” for hind-limb paralysis, “4” for full paralysis, and “5” for deceased. The experiment was single-blinded.

### Tumor Cell Infectivity Assay

JC cells were grown to 70% confluence in 10 cm tissue culture dishes. Cells were infected with VSV23, VSVST, VSVXN2 or VSVwt at MOI=1.0 and incubated for 8 hours at 37° C. and 5% CO₂. Digital photographs were then taken using an Olympus BH2-RFCA microscope (Olympus, Center Valley, Pa.). BHK-21 cells were grown to 70% confluence in 10 cm tissue culture dishes. Cells were infected with VSV23, VSVST, VSVXN2 or VSVwt at MOI=0.01 and incubated overnight at 37° C. and 5% CO₂. Uninfected cells were used as a control. Upon detection of cytopathic effect (CPE), 10 μl of supernatant from each group was then transferred to an individual well of L929 cells that had been grown to 70% confluence. Samples were incubated overnight at 37° C. and 5% CO₂, 10 μl of supernatant from each group was again transferred to an individual well of NB41A3 cells that had been grown to 70% confluence. Cells were visually monitored for signs of CPE for 8 hours and digital photographs were taken.

In vitro Detection of Apoptosis in Mammary Derived Tumor Cells

JC cells were seeded in 96-well plates at a concentration of 4.5×10⁴ and incubated overnight at 37° C., 5% CO₂. Six replicate wells were used for each treatment condition and time point. Cells were then infected at M01=3.0 with VSV23, VSVST, VSVXN2, or VSVwt and incubated at 37° C. 5% CO₂ for 3, 6, 9, 12, 18, or 24 hours. Mock infected cells were used as a negative control. The TACS MTT Cell Proliferation Assay (R&D Systems Minneapolis, Minn.) was used per manufacturer's instructions to conduct the assay. Samples were read at 540 nM on a Biorad 550 series microplate reader.

In vivo Treatment of Mammary Derived Tumors

Cohorts of N=4, six-week old male BALB/c mice were injected subcutaneously on the left dorsal flank with 1×10⁷ JC cells suspended in 40 μl sterile HBSS. Animals were monitored for solid tumor development. Ten days post-implantation, tumors were injected with 1×10⁷ pfu of VSV23, VSVST, or VSVXN2 diluted in 40 μl of PBS or vehicle alone. Viral treatments were repeated on days 3 and 5 after the initial treatment. All viral doses were delivered to four distinct quadrants of the tumor. Tumors were measured daily using 0-150 mm digital calipers (Mitutoyo USA, Aurora, Ill.). Tumor size was calculated using the equation (length/2)²×(width).

### Confocol Microscopy of Immune Cell Infiltration of Tumors

Fourteen days after viral treatment was initiated, animals were arterially perfused with HBSS and tumors were surgically removed. Whole tumors were frozen in Tissue-Tek OCT compound (Sakura, Torrance, Calif.), sliced into 18 μm thick sections using the Leica CM1850UV Cryostat (Leica, Bannockburn, Ill.) and placed on poly-L-lysine-coated slides. Sections were fixed in 4% paraformaldehyde for 10 minutes. The sections were then washed twice with PBS and incubated in goat-α mouse IgG for 45 minutes when necessary. Sections were then incubated in PBS w/Blotto for 45 minutes. The slides were once again washed with PBS and incubated overnight in primary antibodies. Slides were then washed with PBS and incubated in secondary antibody for 45 minutes. After incubation with secondary antibody, the sections were washed with PBS and mounted with Vectorshield Mounting Medium (Vector Laboratories, Burlingame, Calif.). Antibodies are detailed in Table 3. Slides were covered with number 1.5 cover slips (Fisher; Waltham, Mass.), and viewed using a Leica SP5 confocal microscope at 400× magnification. Images are typical of 3 sections of 3 separate tumors for each treatment group

### CTL Assessment of Long-Term Memory Responses Against Tumors

Fourteen days after viral treatment was initiated, spleens from individual mice were harvested, teased into a single cell suspension, and resuspended in MEM, 10% FBS, 1% Pen-Strep. JC cells were used as target cells and plated in 96 well V-bottom plates at 1×10⁴ cells per well. Responder cells from individual mice of each treatment group were added to target cells in triplicate at effector to target ratios of 100:1, 50:1, 25:1, and 12:1. Plates were centrifuged for 5 minutes at 200×g to improve cell contacts and incubated for four hours at 37° C., 5% CO₂. The CytoTox 96™ non-radioactive cytotoxicity kit (Promega, Madison, Wis.) was used per manufacturer's instructions to determine T cell mediated cytolytic activity. Colomeric results were detected with a Biorad 550 series microplate reader (Hercules, Calif.) at 490 nm. Results are representative of three replicate experiments.

### Statistical Analyses

All experimental samples were prepared in triplicate, in at least three separate experiments. Data points representing more than two standard deviations from the mean, or within two standard deviations of background, were culled from data sets. Sample t-values were calculated using Satterthwaite's method for independent samples of unequal variances, and hypothesis testing was employed to determine whether or not quantities (e.g. ³⁵S:³²P ratios) were equal; yielding p-values indicative of these tests. All error bars represent 95% confidence intervals of a particular data set, unless otherwise stated.

### Example 2

### Construction and Sequence of VSV23, VSVST, and Insertion of Restriction Site for Addition of Pathogen Genes

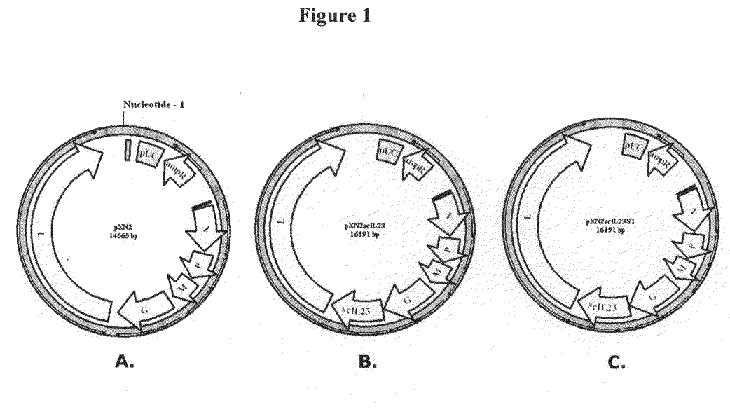

The virus backbone into which IL23 single chain p40 and p19 subunits linked with a spacer peptide [(Gly₄Ser)₃] is introduced is referred to as VSVXN2 (FIG. 1A, showing pXN2 vector) and is described in U.S. Pat. No. 7,153,510 to Rose et al., which is hereby incorporated by reference in its entirety. A novel recombinant vesicular stomatitis virus (VSV) expressing a cytokine, single chain IL23 p40 and p19 subunit, VSV23 (FIG. 1B, showing pXN2-IL23 vector) was created and its biological functions were assayed using a variety of tests. A control virus (VSVST) was also prepared which has the amber mutations introduced in the coding sequence of IL23 (FIG. 1C, showing pXN2-IL23ST vector). This results in the absence of production of IL23. In many studies, additional controls have been introduced, including wild type VSV (VSVwt), Indiana serotype, San Juan strain.

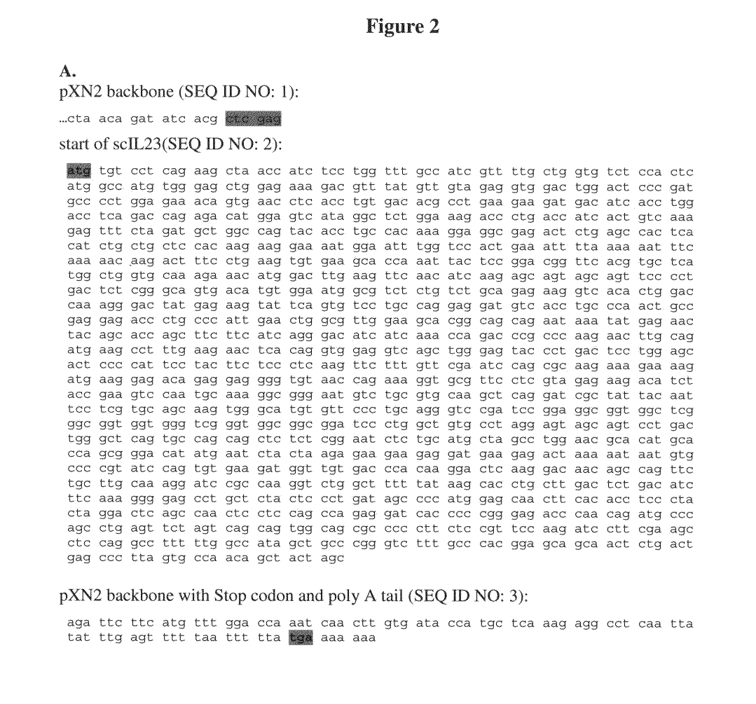

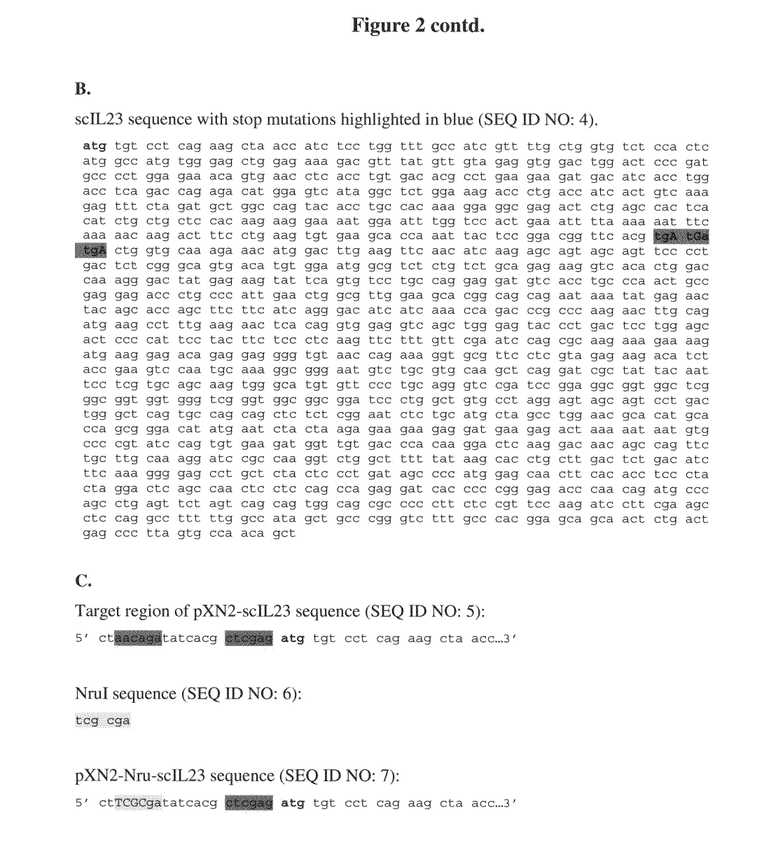

As shown in FIG. 2A, single chain IL23 (scIL23) which includes the p40 and p19 subunits linked by a short peptide [(Gly₄Ser)₃] was isolated by PCR from the pCEP4-scIL231g plasmid (Belladonna et al., “IL-23 and IL-12 Have Overlapping, But Distinct, Effects on Murine Dendritic Cells,” J. Immunol. 168(11):5448-5454 (2002), which is hereby incorporated by reference in its entirety). This reaction removed the Ig region from the 3′ end of the plasmid and introduced Xho I restriction site (red highlighted text) at the 5′ end of the scIL23. Primers utilized are detailed in Table 4. The isolated fragment was subsequently digested and ligated into the pXN2 backbone that had been digested with Xho I and Nhe I. The reaction produced the pXN2-scIL23 plasmid used for VSV23 rescue (Lawson et al., “Recombinant Vesicular Stomatitis Viruses From DNA,” Proc. Nat'l. Acad. Sci. USA 92(10):4477-81 (1995), which is hereby incorporated by reference in its entirety).

Single chain IL23 sequence was mutated using the QuikChange® XL Site-Directed Mutagenesis Kit per manufacturers instructions. See FIG. 2B. Briefly, the scIL23 fragment was ligated into the pSP73 intermediary vector using Kpn I and Xba I restriction sites. This plasmid was subjected to PCR with the blue highlighted font indicating the target sequences and the bold letters indicating the 3 mutated nucleotides resulting in 3 stop codons. The mutated plasmid was then rescued from XL10-gold ultracompetent cells and subjected to PCR to introduce XhoI and Spe I restriction sites at the 5′ and 3′ ends of the scIL23, respectively. The plasmid was isolated and subsequently digested and ligated into the pXN2 backbone that had been digested with Xho I and Nhe I. The reaction produced the pXN2-scIL23ST plasmid used for VSVST rescue and is identical to the pXN21L23 plasmid except for the point mutations. To permit for insertions of DNA encoding pathogenic genes, VSV23 was modified with the creation of a novel Nru I site (yellow-highlighted text; FIG. 2C).

TABLE 4
Primers for Recombinant VSV Production

| Primer | | |
|---|---|---|
| Designation | Sequence (5′ to 3′) | |
| J Mp40XhoI | tagtcctcgagatgtgtcctcagaagctaaccatct | (SEQ ID NO: 14) |
| JMp1 9SpeI | tatgaactagtctaagctgttggcactaagggct | (SEQ ID NO: 15) |
| JM033p40MutF | actccggacggttcacgtgatgatgactggtgcaaagaaacatgg | (SEQ ID NO: 16) |
| JM034p40MutR | ccatgtttctttgcaccagtcatcatcacgtgaaccgtccggagt | (SEQ ID NO: 17) |

rVSVs were rescued in BHK-21 cells using the previously described reverse genetics method (Lawson et al., “Recombinant Vesicular Stomatitis Viruses From DNA,” Proc. Nat'l. Acari Sci. USA 92(10):4477-81 (1995), which is hereby incorporated by reference in its entirety). Briefly, cells were infected with vaccinia virus expressing the T7 RNA polymerase, then transfected with pXN2-scIL23, pXN2-scIL23ST, or pXN2 to produce VSV23, VSVST, and VSVXN2 respectively. In addition, plasmids encoding N, P and L proteins were co-transfected using LipofectAMINE 2000 (Invitrogen, Carlsbad, Calif.). Vaccinia virus was removed by filtration through a 0.20 μm filter after 48 hours of incubation. Filtrate was added to fresh BHK-21 cells. Subsequently, individual clones were plaque purified and used for production of viral stocks. Titers of rVSV were determined by plaque assay on L929 cells. VSV Indiana strain, San Juan serotype, was originally obtained from Alice S. Huang (then at The Children's Hospital; Boston, Mass.).

### Example 3

### VSV23 Infection in vitro Results in Production and Secretion of IL23

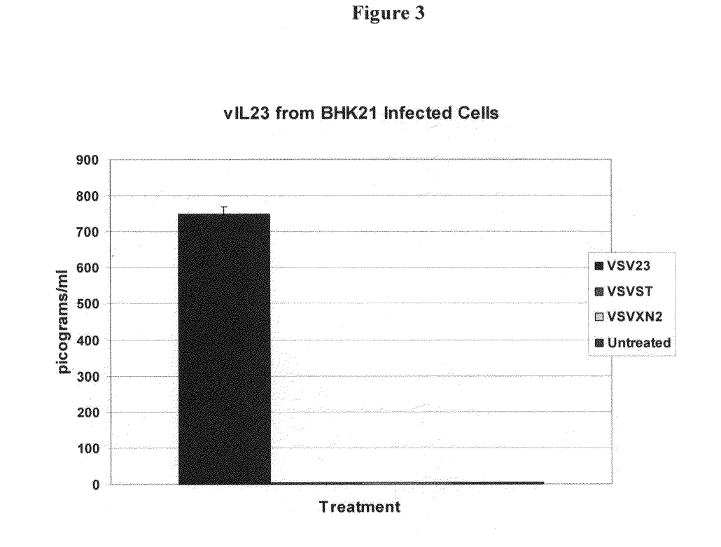

Supernatant of cells infected with the panel of viruses (VSV23, VSVST, VSVNX2) were assayed for the presence of IL23 by ELISA (FIG. 3) and by bioassays. Three assays to examine cytokine production were performed on supernatants obtained from BHK21 cells infected with VSV23 and other viruses in the panel as follows: a) secondary activation of neuronal cells to produce nitric oxide (NO); b) induction of IFN-γ mRNA production by primary murine splenocytes; and c) an ELISA to detect secreted IL23. Virally infected supernatant was harvested and subjected to UV inactivation to inactivate the virus. Supernatant from uninfected BHK21 cells was used as a negative control. Samples were then subjected to the Quantikine Mouse IL12/IL23 p40 (non allele-specific) Immunoassay ELISA kit from R&D Systems. Supernatant from VSV23 infected cells contained 750 pg/ml of the p40 subunit. The experiment indicates that there are no detectable levels of p40 secreted by cells infected with VSVST or VSVXN2. BHK21 cells do not produce IL23 and as expected control samples did not produce detectable levels of the cytokine component.

Only VSV23-infected cells secreted biologically detectable and active IL23. The data shown in FIG. 3 unambiguously indicate that VSV23 infection (and only that virus infection) results in the release of immunologically recognized IL23.

### Example 4

### VSV23 is not Attenuated for Growth in Established Cell Lines In Vitro

VSV23 and the other viruses of the present invention were tested for the ability to replicate in vitro in multiple cell lines (L929, a Murine Adiposite line; BHK21, a baby hamster kidney epithelial cell line; NB41A3, a Murine Neuroblastoma.

One-step growth curve experiments conducted with L929 cells indicated that there was little difference in the growth kinetics of rVSVs and VSVwt. This should not be the case in cells that are responsive to IL23. In order to test this hypothesis, both non-responsive L929 cells and responsive NB41A3 cells were infected with rVSVs or VSVwt and supplemented with rIL23 or PBS as a control. Plaque assays at varying time points were conducted to indicate if there is an alteration in viral titers due to the activity of IL23. The mechanism of attenuation is hypothesized to be nitric oxide (NO). NO is a potent antiviral component of the immune response in the CNS (Bi et al. “Vesicular Stomatitis Virus Infection of the Central Nervous System Activates Both Innate and Acquired Immunity,” J Virol 69:6466 (1995); Ireland et al. “Gene Expression Contributing to Recruitment of Circulating Cells in Response to VSV Infection of the CNS,” Viral Immunol 19:3 (2006); Reiss et al., “Innate Immune Responses in Viral Encephalitis,” Curr Top Microbiol Immunol 265:63 (2002); Hao et al. “Immune Enhancement and Anti-tumour Activity of IL23,” Cancer Immunol Immunother 55:1426-1431 (2006), which are hereby incorporated by reference in their entirety).

IL23 (whether virally produced or added exogenously) is expected to inhibit viral production in NB41A3 cells. These cells have been shown to produce NO in response to IL23 treatment. No change in viral titers between treatment groups is expected in L929 cells as they are not responsive to IL23.

L929 and NB41A3 cells were grown to 90% confluence and infected at MOI=0.001 in duplicate with VSV23, VSVST, VSVXN2, or VSVwt. One set of each panel was supplemented with 0.25 ng/ml rIL23. Supernatants were harvested from infected cells at 12, 16, 20, and 24 hours post-infection (p.i.). L929 cells were grown to confluence and treated with serial dilutions of virally infected supernatants from each time point and allowed to incubate at 25° C. for 30 minutes. Viral titers were then assessed using the plaque assay technique. Data shown are from three replicate experiments.

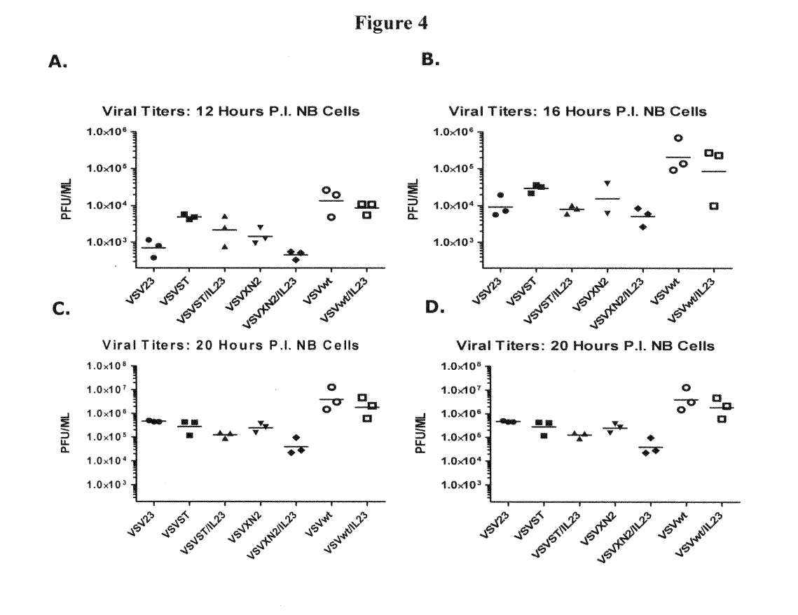

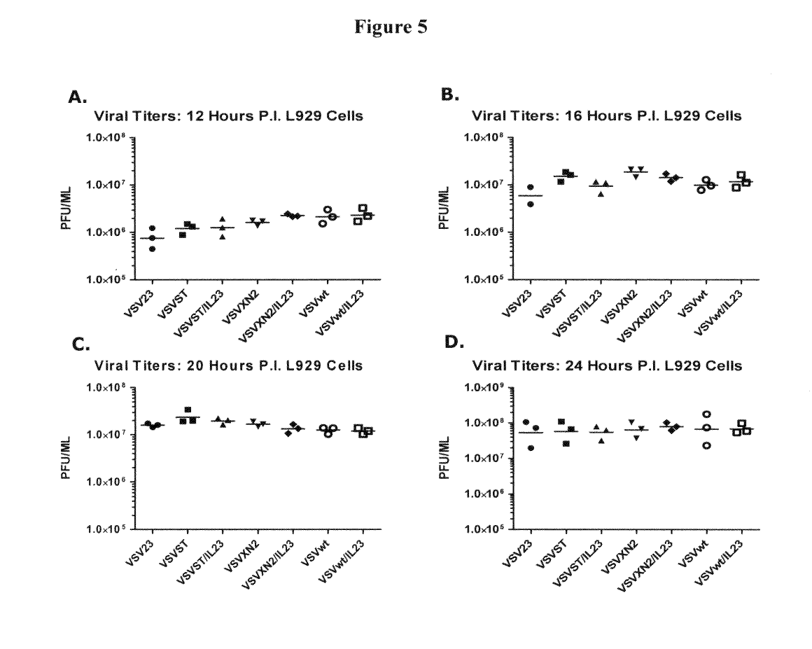

IL23 induces a modest reduction in viral titers in NB41A3 cells (FIG. 4). Viral titers are reduced by 50% to one log at all time points except for the VSVST infection at 24 hours. Almost no difference in viral titers was detected in L929 infected cells regardless of treatment (FIG. 5).

This data indicates that IL23 results in some inhibition of viral replication in IL23 responsive cells. Previous experiments showed that supernatants harvested from VSV23 infected BHK-21 cells and UV treated to inactivate viral particles induced NO in NB41A3 cells. Cells exogenously treated with rIL23 in this experiment also produced NO. It is hypothesized that the production of NO in response to vIL23 or rIL23 is responsible for the changes seen in viral titers in NB41A3 infected cells. This hypothesis can be tested by conducting Greiss assays on supernatants from infected and treated cells. Additionally, it is possible that increasing the dose of exogenously added rIL23 would result in greater inhibition of viral production through enhanced induction of NO. This hypothesis can also be tested by the Greiss and plaque assay techniques. It should be noted that experiments have consistently shown lower titers of VSV produced by NB41β3 cells compared to L929 cells. The discrepancy in viral titers seen in this experiment between the cell lines matched expectations.

### Example 5

### Attenuation of VSV23 for CNS Pathology and Viral Encephalitis

It is essential to understand and study the pathogenesis of lethal VSV encephalitis in mice. Any new vaccine or oncolytic virus must have complete attenuation for injuring hosts while attempting to provide beneficial activities. Therefore, the ability of VSV23 to cause disease in mice when administered intranasally, the route which leads to viral encephalitis, was studied. VSV23 was compared with the recombinant VSVXN2 and wild type VSV as well as the VSVST viruses for the ability to cause illness and death, as well as more subtle indicators of infection.

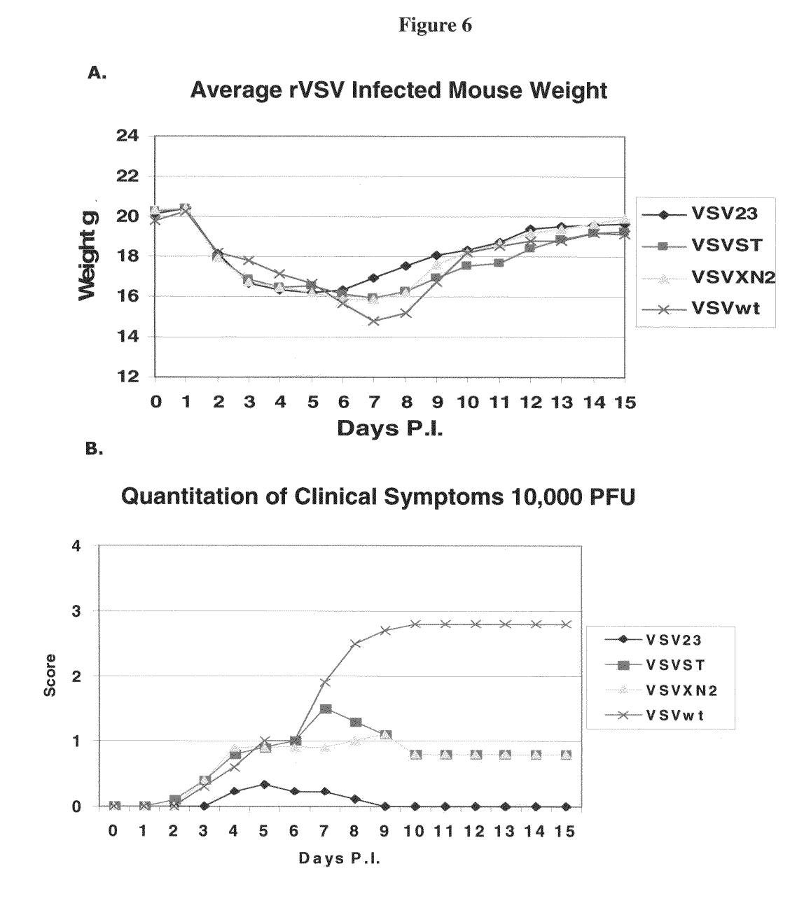

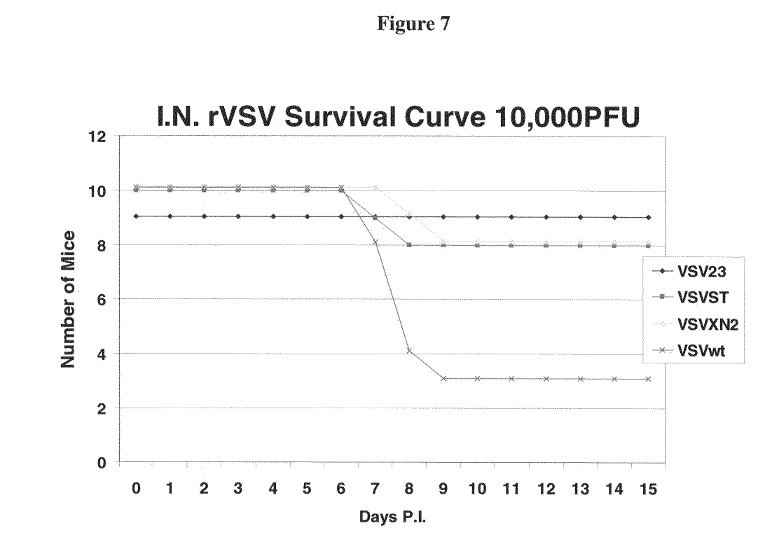

Morbidity is a complex cluster of symptoms associated with illness. In mice it is measured by several means: weight loss, changes in grooming and activity, hind-limb paralysis. Some of these characteristics are subjective, but others can be readily quantitated. In FIG. 6, both weight loss (quantitative, left) and synthesis of the subjective values (right) are shown for the cohorts of mice infected by the viruses over a 2 week observation period. Mortality is shown in FIG. 7 for the same group of mice during the same period. No mice infected with VSV23 died in two separate infections, while all other viruses induced viral encephalitis and resulted in some mortality.

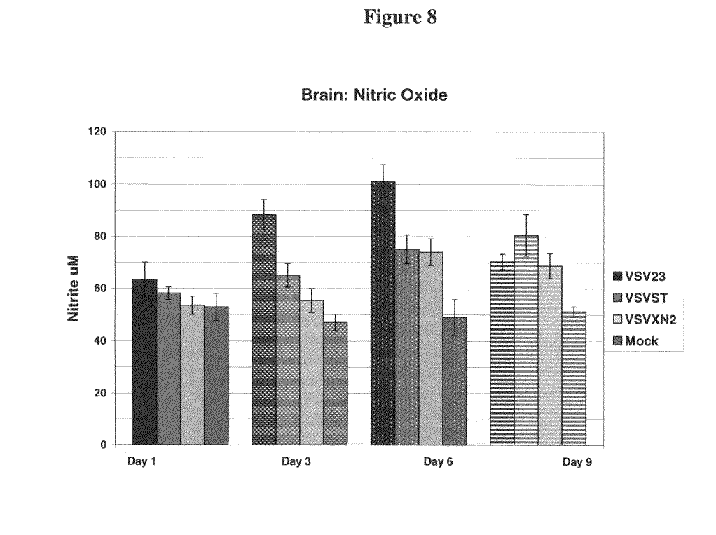

VSV23 is highly attenuated when introduced intranasally to mice in the viral encephalitis model. Groups of mice were administered VSV23 or the other viruses in the panel. In assays of morbidity (FIG. 6), mortality (FIG. 7), induction of nitric oxide production which is an essential component promoting recovery in the innate immune response to infection (FIG. 8), viral replication in the CNS (Table 5), and immunohistologic indicators of pathogenesis, VSV23 was easily distinguished from the other viruses and is highly attenuated in the sensitive encephalitis model.

An important question is whether virus replicates in the CNS of VSV23-infected mice like it does in mice infected with XN2 or WTVSV. The data from two experiments to check the replication are shown in Table 5. Although some mice had very low titers of VSV23 in their brains following intranasal infection (for half the mice, VSV23 was below the level of detection, ˜200 pfu/hemisphere), this was not associated with morbidity or mortality (FIGS. 7 and 8).

TABLE 5
VSV titers in CNS homogenates of mice infected intranasally.

| VSV23 | Day | Mouse 1 | Mouse 2 | Mouse 3 | Mouse 4 | Mouse 5 | Mouse 6 | Mean* | G Mean* |
|---|---|---|---|---|---|---|---|---|---|
| Exp. 1: | 1 | ≦200 | ≦200 | ≦200 | 667 | ≦200 | 1000 | 833.5 | 816.7 |
| | 3 | 7833 | 58333 | 3833 | 2500 | 8667 | N/A | 16233.2 | 8238.31 |
| | 6 | ≦200 | 22500 | ≦200 | ≦200 | 12333 | 11833 | 15555.33 | 14863.3 |
| | 9 | ≦200 | ≦200 | ≦200 | ≦200 | ≦200 | ≦200 | Below | Below |
| | | | | | | | | Detection | Detection |
| Exp. 2: | 1 | 3167 | 4667 | ≦200 | ≦200 | 4833 | 1657 | 3583.5 | 3303.39 |
| | 3 | ≦200 | 8667 | 2333 | 93333 | 3000 | N/A | 26833.25 | 8674.31 |
| | 6 | ≦200 | 500 | 7333 | ≦200 | 1667 | ≦200 | 3166.67 | 1828.36 |
| | 9 | ≦200 | ≦200 | ≦200 | ≦200 | ≦200 | ≦200 | Below | Below |
| | | | | | | | | Detection | Detection |
| VSVST | Day | Mouse 1 | Mouse 2 | Mouse 3 | Mouse 4 | Mouse 5 | Mouse 6 | Mean | G Mean |
| Exp. 1: | 1 | 4170 | 3550 | 2167 | 1000 | 2333 | 330 | 2258.33 | 1706.51 |
| | 3 | 142500 | 14000 | 75000 | 55000 | 28334 | N/A | 62966.8 | 47155.78 |
| | 6 | 383333 | 550000 | 4666 | 250000 | 733333 | 25000 | 331388.67 | 188663.02 |
| | 9 | 196666 | ≦200 | 28333 | ≦200 | ≦200 | N/A | 111499.5 | 71963.92 |
| Exp. 2: | 1 | 3833 | 1833 | 3833 | 6167 | 5833 | 28333 | 8305.33 | 5492.24 |
| | 3 | 10500 | 16667 | 150000 | 68333 | 28333 | 41667 | 52583.33 | 35835.12 |
| | 6 | 26867 | 500000 | 183333 | 60000 | 250000 | 11667 | 171944.5 | 86803.35 |
| | 9 | 1833 | 1833 | 2000 | 400 | 6167 | ≦200 | 2446.6 | 1753.47 |
| VSVXN2 | Day | Mouse 1 | Mouse 2 | Mouse 3 | Mouse 4 | Mouse 5 | Mouse 6 | Mean | G Mean |
| Exp. 1: | 1 | 2433 | 11000 | 5167 | 1333 | 9167 | N/A | 5820 | 4421.46 |
| | 3 | 9500 | 18000 | 105000 | 45000 | 135000 | N/A | 62500 | 40506.51 |
| | 6 | 2466666 | 200000 | 40000 | 50000 | 166667 | 766666 | 614999.83 | 223925.92 |
| | 9 | 146667 | 500 | ≦200 | 4833 | 75000 | N/A | 56750 | 12768.65 |
| Exp. 2: | 1 | 21667 | 8000 | 20667 | 13250 | 10500 | 10833 | 14152.83 | 13245.04 |
| | 3 | 30000 | 36687 | 11167 | 17833 | 55000 | N/A | 30133.4 | 26072.58 |
| | 6 | 700000 | 216667 | 233333 | 171667 | 750000 | 143333 | 269166.67 | 225250.21 |
| | 9 | 15167 | 2833 | 817 | 1667 | 2833 | ≦200 | 4663.4 | 5932.95 |
| VSVwt | Day | Mouse 1 | Mouse 2 | Mouse 3 | Mouse 4 | Mouse 5 | Mouse 6 | Mean | G Mean |
| Exp. 1: | 1 | 2500 | 2333 | 23333 | 1667 | 18333 | 10500 | 9777.67 | 5934.22 |
| | 3 | 500000 | 450000 | 383333 | 130000 | 483333 | N/A | 389333.2 | 352200.28 |
| | 6 | 2466666 | 2300000 | 3200000 | 816606 | 216657 | 2133333 | 1855545.33 | 1378188.72 |
| | 9 | 250000 | 366666 | ≦200 | ≦200 | 58333 | N/A | 224999.67 | 174867.19 |

Groups of 6 BAlB/cAnTac male mice were infected with the panel of viruses, in two separate experiments. At the days indicated after infection, individuals were sacrificed and brains were divided, sagitally. One half was homogenized, serially diluted,and assayed by plaque assay on L929 cells for the presence of VSV. Data are presented as the average of 3 replicate samples for each individual and both average and geometric mean or the group. The lower limit of detection was 200 pfu/half-brain.

### Example 6

### Production of Nitrous Oxide (NO) in vivo During Infection of the CNS

The construction of this recombinant virus was done in order to take advantage of local IL23 expression. IL23 uses the same signaling receptor chain as IL12 (Kastelein et al., “Discovery and Biology of IL23 and IL27: Related but Functionally Distinct Regulators of Inflammation,” Annual Review of Immunology 25:221-42 (2007), which is hereby incorporated by reference in its entirety), which applicants have shown in many published studies induces the production of nitric oxide (NO) in the CNS and promotes survival and recovery from VSV encephalitis (Ireland et al., “Interleukin (IL)-12 Receptor β1 or IL12 Receptor β2 Deficiency in Mice Indicates that IL12 and IL23 are not Essential for Host Recovery from Viral Encephalitis,” Viral Immunol 18:397-402 (2005); Ireland et al., “Expression of IL12 Receptor by Neurons,” Viral Immunol. 17:41122 (2004); Ireland et al., “Delayed Administration of Interleukin-12 is Efficacious in Promoting Recovery from Lethal Viral Encephalitis,” Viral Immunol. 12:35-40 (1999); Komastu et al., “IL12 and Viral Infections,” Cytokine Growth Factor Rev 9:277-85 (1998); Komatsu et al., “Regulation of the BBB during Viral Encephalitis: Roles of IL12 and NOS,” Nitric Oxide 3(4):327-39 (1999); Bi et al., “IL12 Promotes Enhanced Recovery from Vesicular Stomatitis Virus Infection of the Central Nervous System,” J. Immunol. 155(12):5684-9 (1995), which are hereby incorporated by reference in their entirety). Therefore, it was important to determine if this virus was able to induce the production of NO in vivo during infection of the CNS. Mice were infected intranasally with the panel of viruses and on days 1, 3, 6, and 9 after infection, homogenates of brain tissue from individuals were examined for the presence of NO by a colorometric test, the Greiss assay. As shown in FIG. 8, VSV23 induced increased NO production on days 1, 3, and 6, post-infection, despite the fact that this virus did not induce death or symptoms of illness (FIGS. 6 and 7).

These studies have demonstrated that VSV23 does not cause death or illness and readily induces the production of NO.

### Example 7

### Morbidity and Mortality Assessment at 1×10⁶ pfu

VSV infection of the CNS of mice results in encephalitis with the symptoms of lack of grooming behavior, weight loss, hind-limb paralysis, and death. Upon intra nasal (i.n.) administration, the virus infects olfactory sensory neurons in the nasal turbinates and spreads along the olfactory nerve to the olfactory bulb. From the olfactory bulb, infection spreads caudally through synapses, and once at the olfactory ventricle, in cerebral spinal fluid to motor neurons in the lumbar-sacral spinal cord, giving rise to the symptoms of encephalitis and hind-limb paralysis in the infected animal. The cause of death from VSV encephalitis may be due to break-down of the blood brain barrier, involvement of higher centers regulating respiration and heart-beat, or hind-limb paralysis (Forger et al. “Murine Infection by Vesicular Stomatitis Virus Initial Characterization of the H-2^{d} System,” J Virol 65:4950 (1991); Huneycutt et al. “Distribution of Vesicular Stomatitis Virus Proteins in the Brains of BALB/c Mice Following Intranasal Inoculation: An Immunohistochemical Analysis,” Brain Res 635:81 (1994); Lundh et al., “Selective Infections of Olfactory and Respiratory Epithelium by Vesicular Stomatitis and Sendai Viruses,” Neuropathol Appl Neurobiol 13:111 (1987), which are hereby incorporated by reference in their entirety). Disease progression can be determined by monitoring weight loss, alterations in grooming and behavior, paralysis, and death. Previous data has shown that VSV23 is attenuated compared to control viruses and VSVwt at a dose of 1×10³. Increasing the infectious dose administered to subjects will help determine the degree of attenuation. Pilot studies have shown that mortality is detected in VSV23 infected mice at a dose of 1×10⁶.

VSV23 infected mice are expected to exhibit lower levels of morbidity and mortality compared to VSVST and VSVXN2. VSVST and VSVXN2 infected mice are expected to show comparable levels of morbidity and mortality.

Cohorts of 10, 6-week old BALB/c mice were infected intranasally with 1×10⁶ pfu of VSV23, VSVST, or VSVXN2 and monitored for 15 days. Mice were weighed daily to monitor for weight loss and health. Hind-limb paralysis or weight loss that exceeded 30% of starting weight were considered end points for the experiment. Subjects were scored on a subjective 6 point scale (0-5): “0” for no symptoms, “1” for lack of grooming behavior, “2” for hunched and severely lethargic mice, “3” for hind-limb paralysis, “4” for full paralysis, and “5” for deceased. The experiment was blinded at the time of infection. One independent party diluted virus, while another color coded the samples. The color code was broken after 15 days of monitoring the animals. The experiment was repeated twice.

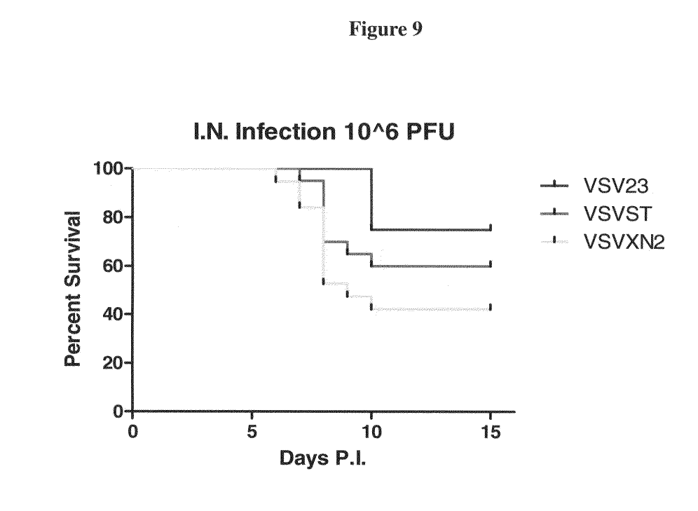

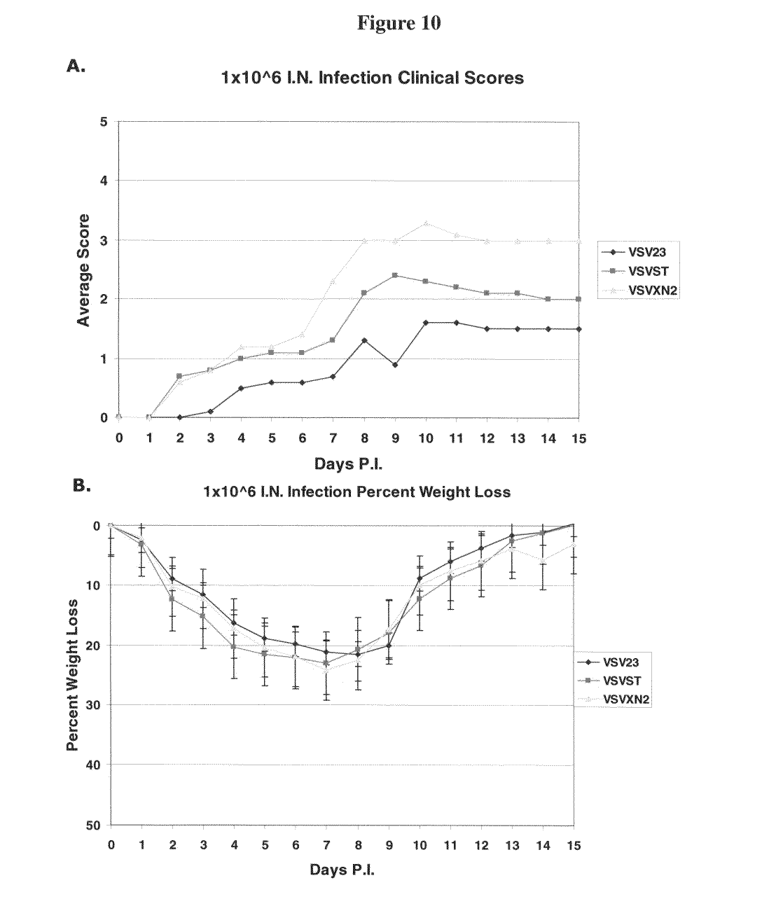

VSV23 infection resulted in 25% mortality. Infection with VSVST and VSVXN2 resulted in 40% and 58% mortality, respectively (FIG. 9). Kaplan-Meier survival curve analysis utilizing the one-tail p value indicates that VSV23 is different from the other viruses by p<0.05. The non-parametric Kruskal-Wallis analysis of symptom data indicates a significant difference in clinical scores among the groups; p<0.05. Standard deviations of average percentage weight loss for all groups indicate no significant difference in weight among all infection groups (FIG. 10).

Decreased morbidity and mortality in VSV23 infected mice compared to control viruses indicates that vIL23 induces enhanced innate immune responses resulting in decreased morbidity and mortality confirming applicants' expectations. The dose of 1×10⁶ was chosen based on pilot studies utilizing increasing log doses to determine the minimum pfu of VSV23 that could induce mortality. Results of IHC studies from animals infected with 1×10³ pfu of rVSVs indicate that there is upregulation of Nitric oxide synthase type II (NOS II) in microglia and macrophages one day p.i in VSV23 infected mice. Greiss assays to determine the amount of NO in the CNS of animals infected with 1×10³ and 1×10⁶ pfu of rVSVs indicates increased levels of NO in VSV23 infected mice at day 3 p.i. These data indicate that increased levels of NO may be accountable for the attenuation of VSV23 seen at all doses examined during this project when compared to other viruses. While the ability of VSV23 to induce mortality is a point of concern, the dose is much higher than that of VSVwt which may induce mortality at doses as low as 1×10².

### Example 8

### vIL23 Results in Attenuated VSV in the CNS at 1×10⁶ pfu

When VSV is administered to animals intranasally, infection is established in the olfactory bulb. The virus then spreads caudally through the brain resulting in VSV induced encephalitis. Experiments have indicated that VSV23 is attenuated in the CNS at 1×10³ pfu. To determine the extent and mechanism of attenuation, the infection was performed at 1×10⁶ pfu. Viral titers in the CNS were determined at days 1 and 3 p.i. NO levels in the CNS were determined by the Greiss assay.

Morbidity and mortality data indicate that VSV23 is attenuated at 1×10⁶ pfu. VSV23 viral titers are expected to be lower compared to control viruses. NO levels are expected to be significantly higher in VSV23 infected animals compared to control infection due to the previously established activity of vIL23 in the CNS. Cohorts of 6,6-week old male BALB/c mice were infected i.n. with 1×10⁶ pfu of VSV23, VSVST, or VSVXN2. Individuals were sacrificed on days 1 and 3 p.i., and brains were divided sagitally. One half of the brain was homogenized, serially diluted, and subjected to the Greiss and plaque assays, as previously described, to determine NO levels and viral titers in the CNS. Data are presented as the average of 3 replicate samples for each individual and the geometric mean of the cohort. The lower limit of detection for plaque assay was 200 pfu/half-brain.

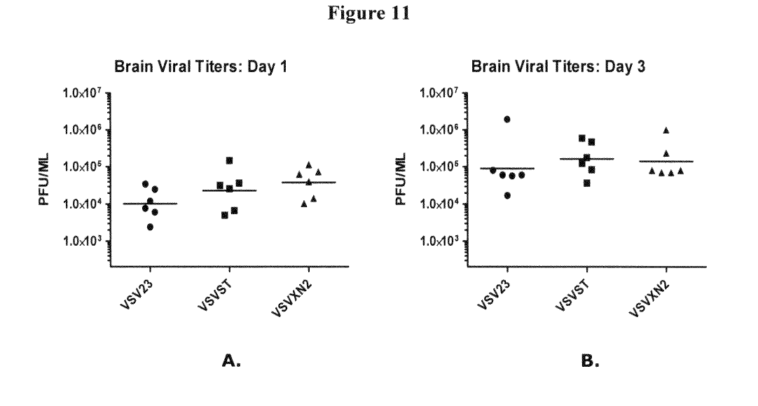

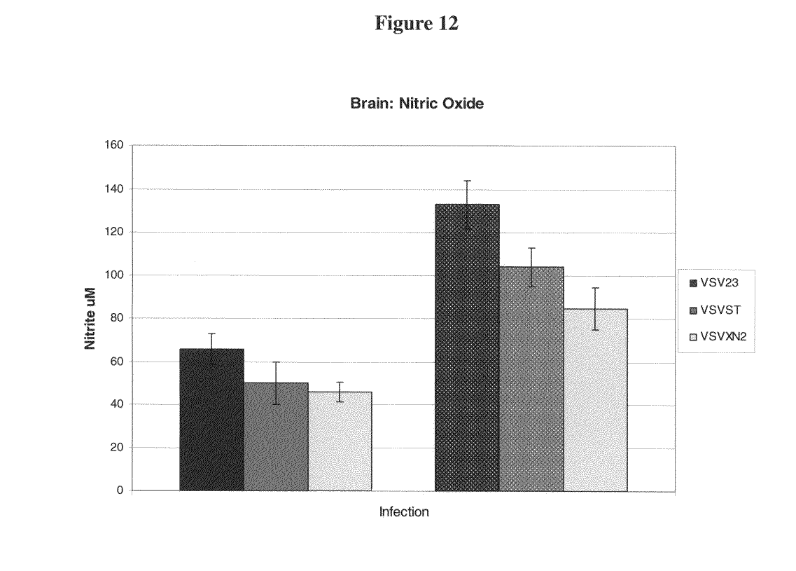

No significant difference in viral titers is detected among rVSV infected animals (FIG. 11). This result did not match expectations. Measurement of NO levels indicates a significant increase in NO in VSV23 infected animals compared to control viruses, p<0.05 as determined by ANOVA analysis (FIG. 12). NO levels on day 3 in VSV23 infected animals were comparable to those seen on day 6 when infected with 1×10³ pfu.

Animals infected with 1×10⁶ pfu of VSV23 exhibit lower levels of morbidity and mortality. However, this does not correlate to decreased viral titers on days 1 and 3 p.i. It is possible that at this early stage in the infection innate immune responses are being overwhelmed, but are subsequently capable of controlling the infection at later time points. Though there is no significant increase in NO levels day 1β.i., by day 3 significantly increased levels are detected. It is hypothesized that the increased levels of NO are a key component of the decreased morbidity and mortality that is characteristic of VSV23 infection. Viral titers at later time points would be expected to begin decreasing except in animals that would eventually succumb to the infection. IHC analysis of brains harvested from mice infected at 1×10⁶ will provide more information on the immune response and spread of the virus during the first 3 days of infection. One hypothesis is that the spread of the virus in the CNS is limited in VSV23 infected animals. This would allow for high viral titers without infection of critical regions of the brain. These studies are currently being conducted.

### Example 9

### VSV23 is Highly Immunogenic for Host Responses and Indistinguishable From Other Viruses not Encoding Secreted IL23

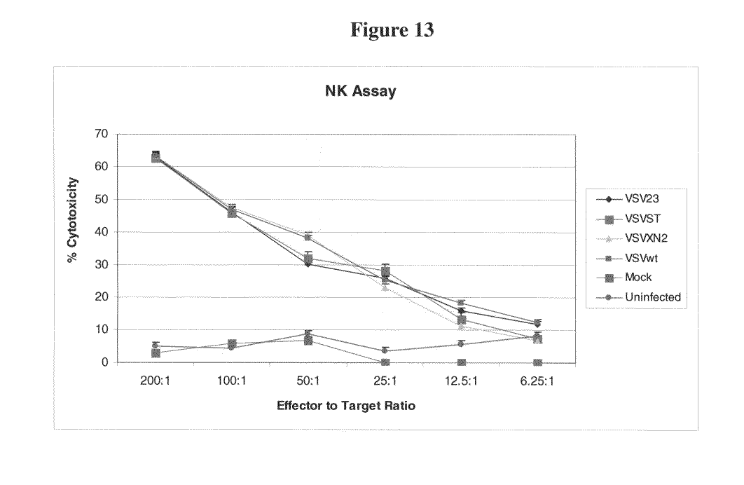

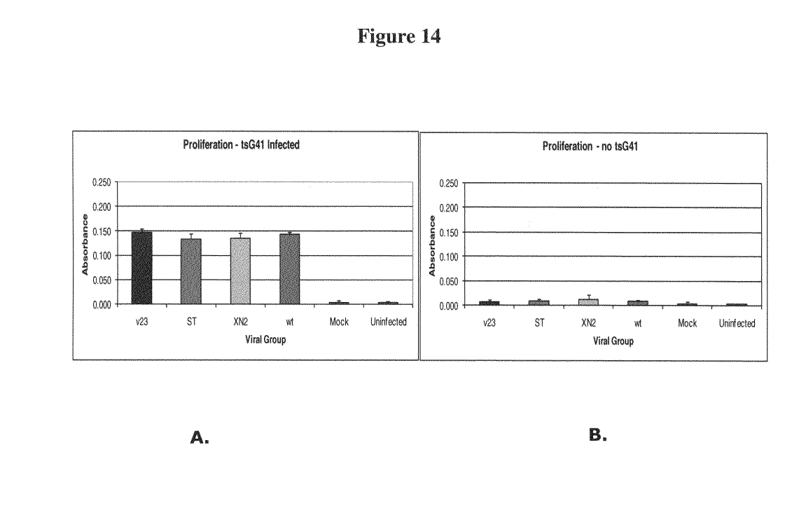

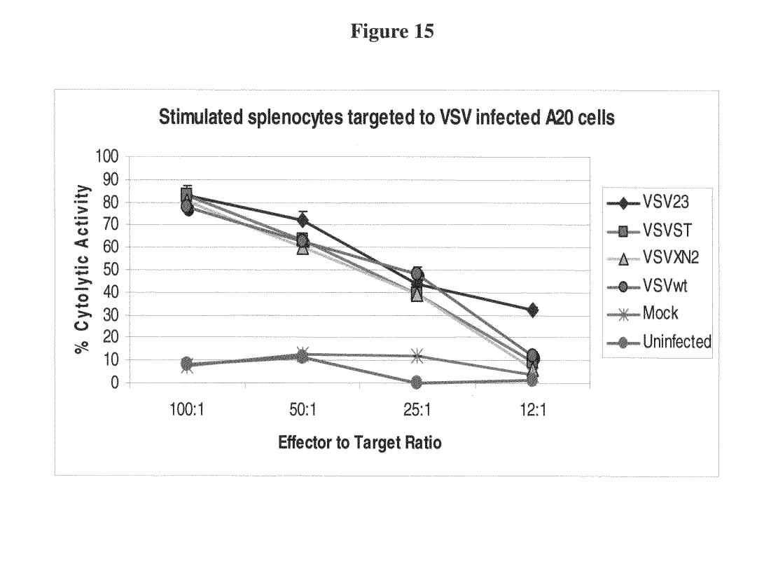

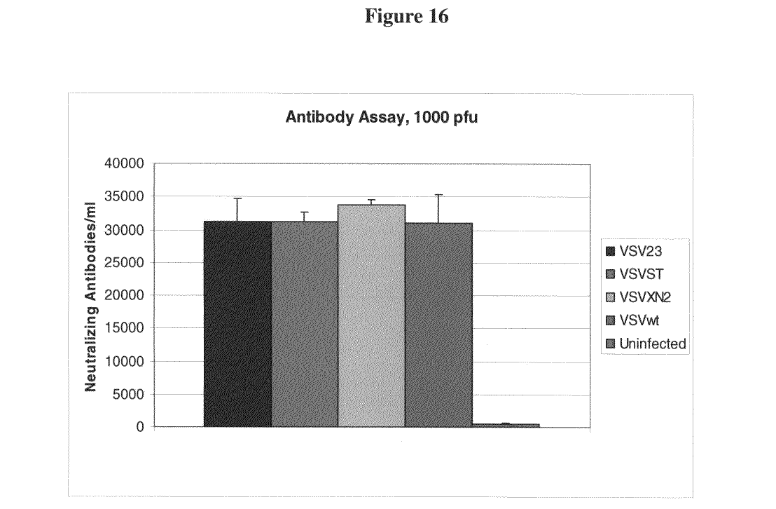

VSV23 is indistinguishable from other viruses not encoding secreted IL23 in immunogenicity. When injected intraperitoneally into BALB/c mice, VSV23 elicited both innate and acquired immune responses comparable to those of VSVST and VSVXN2 viruses. The tested assays include induction of natural killer (NK) cells (FIG. 13), proliferating virus-specific CD4 T cells (FIG. 14), cytolytic T cells (FIG. 15), and production of neutralizing antibody (FIG. 16). Thus, VSV23 is readily able to elicit host responses and these are not statistically different than those responses stimulated by other VSVs tested at the same time.

### Example 10

### Induction of Host Innate and Acquired Immune Responses by VSV23

Simultaneous immunizations of groups of mice with the panel of viruses was performed to determine if VSV23 was immunogenic in vivo following parenteral exposure. A wide variety of assays were done. Viral infection or immunization is one of the most effective ways to induce natural killer cells (NK cells), an innate immune response to infection which is not antigen-restricted or histocompatibility specific (cytolytic T lymphocyte [CTL] responses are highly restricted to their eliciting antigen and MHC) and are assayed ex vivo using the Yac-1 target cell. FIG. 13 clearly demonstrates that the NK responses elicited by VSV23 are indistinguishable from those induced by VSVST, VSVXN2, or wild type VSV.

The ability of viruses to induce memory specific Th1 cell responses is often measured by the induction of antigen-specific proliferating cells. Mice were immunized with the panel of viruses to examine whether the VSV23 or VSVST recombinants were as immunogenic for eliciting memory CD4 virus-specific responses. These viruses were indistinguishable from the gold standard, WT VSV (FIG. 14).

The ability of viruses to elicit host cytolytic T lymphocyte (CTL) responses which control infection and promote recovery is a hallmark of host acquired immunity to infection. Tests were conducted to find if VSV23 immunization was able to induce the differentiation of CTLs specific for VSV. Mice were immunized as above with the panel of viruses. Secondary CTL activity was assayed on VSV infected or uninfected A20 target cells following in vitro culture with either specific antigen (VSVtsG41 infected syngeneic cells) or mock-infected syngeneic cells. As above, VSV23 is immunogenic for induction of CTL responses against VSV (FIG. 15), and indistinguishable from the panel of viruses.

The ability of VSV23 to induce mice to produce neutralizing antibody was tested. The ability to induce the production of neutralizing antibody is critical for protection against secondary viral infections, and an essential characteristic of any vaccine. Groups of mice were infected intranasally with the panel of the viruses, and the surviving individuals were bled 20 days after immunization. The individual serum samples were serially diluted and mixed with 1×10³ pfu of VSV and then plated onto an indicator cell line (L929 cells). In the absence of antibody, viral plaques develop overnight. The limit dilution of serum antibody protecting the indicator cells from VSV infection was determined (FIG. 16). VSV23 was comparable to the other viruses in inducing protective neutralizing antibody.

### Example 11

### Immuno-histochemical (IHC) Analysis of Brain Sections to Monitor Immune Responses

Intranasal infection of mice with VSV results in viral propagation through budding from the basolateral surface of polarized cells and the subsequent establishment of the virus in the olfactory bulb. The virus then spreads caudally through the brain. Innate and adaptive immune responses are mounted against the virus. The spread of the virus and the response of immune cells (such as macrophages and microglia, neutrophils, CD4⁺ and CD8⁺ cells) can be monitored by IHC. Cells producing antiviral proteins such as NOS II can also be detected in this fashion.

Animals infected with VSV23 may exhibit enhanced recruitment of macrophages and neutrophils to the site of infection compared to those infected with VSVST, VSVXN2, and VSVwt (McKenzie et al., “Understanding the IL23-IL17 Immune Pathway,” Trends Immunol 27(1):17-23 (2006); Chen et al., “Anti-IL23 Therapy Inhibits Multiple Inflammatory Pathways and Ameliorates Autoimmune Encephalomyelitis,” J Clin Invest 116(5):1317-1326 (2006), which are hereby incorporated by reference in their entirety). No change in recruitment of CD4⁺ and CD8⁺T cells is expected. Greiss assay data leads to the hypothesis that NOS II will be upregulated more robustly in VSV23 infected animals compared to controls and VSVwt. Expression of NOS I and NOS III may also be enhanced. It is conceivable that the attenuation of VSV23 will result in a rapid clearance of the virus. Subsequently, a robust upregulation of adaptive immune responses that would otherwise be induced by the activity of vIL23 would be prevented. This must be accounted for when analyzing data.

6 week old BALB/c mice were infected intranasally with 1×10³ pfu of VSV23, VSVst, VSVXN2, or VSVwt. Uninfected mice were used as a negative control. Brains were harvested on days 1, 3, 6, and 9 and stored at −80° C. Sagittal sections were cut on a cryostat (20 μm) and sections were fixed in 4% paraformaldehyde for 10 minutes. The sections were then washed twice with PBS and incubated in goat-α mouse IgG for 45 minutes. Sections were then incubated in PBS w/Blotto for 45 minutes. The slides were once again washed with PBS and incubated overnight in primary antibodies. Slides were then washed with PBS and incubated in secondary antibody for 45 minutes. Antibody treatments are shown in Table 6.

TABLE 6
Primary and Secondary Antibodies used

| Primary | | | | |
|---|---|---|---|---|
| Antibody | Specificity | Dilution | Secondary | Dilution |
| rat α-mouse | Mouse | 1:200 in PBS | goat α-rat Alexa | 1:100 in PBS & |
| CD11b | Macrophages & | | Fluor ® 488 | Blotto |
| | Microglia | | | |
| rat α-mouse | Mouse | 1:200 in PBS | goat α-rat Alexa | 1:100 in PBS & |
| RB68C5 | Neutrophils | | Fluor ® 488 | Blotto |
| rat α-mouse | Mouse CD4 & | 1:200 in PBS | goat α-rat Alexa | 1:100 in PBS & |
| L3T4 | CD8 cells | | Fluor ® 488 | Blotto |
| rat α-mouse | | | | |
| Ly-2 | | | | |
| rabbit α- | Mouse NOS I | 1:500 in PBS | donkey α-rabbit | 1:100 in PBS & |
| mouse NOS I | | | Alexa Fluor ® 546 | Blotto |
| rabbit α- | Mouse NOS II | 1:100 in PBS | donkey α-rabbit | 1:100 in PBS & |
| mouse NOS II | | | Alexa Fluor ® 546 | Blotto |
| rabbit α- | Mouse NOS III | 1:200 in PBS | donkey α-rabbit | 1:100 in PBS & |
| mouse NOS III | | | Alexa Fluor ® 546 | Blotto |

After incubation with secondary antibody, the sections were washed with PBS and Vector Shield Mounting Medium with DAPI was added. Digital photographs were taken on an Olympus BH2-RFCA microscope.

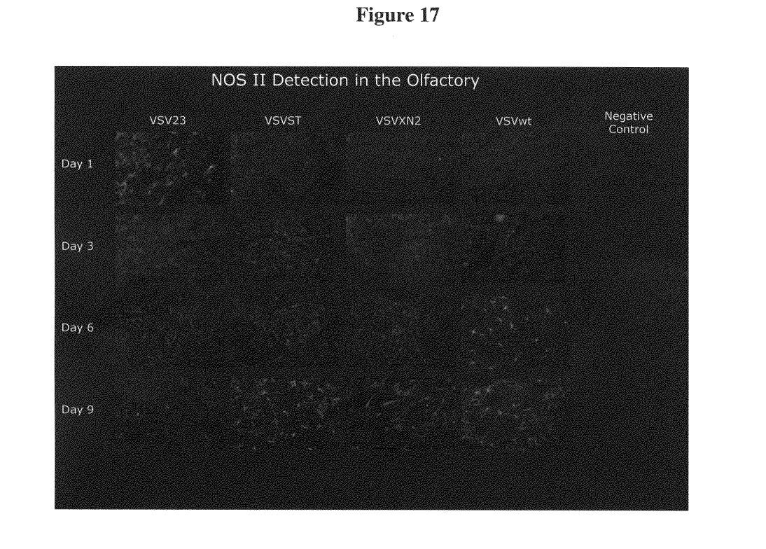

Little to no induction above that of the basal level of NOS I and NOS III was detected in any of the infection groups. VSV23 infection induces cells to express NOS II on day 1 p.i.; however, this induction is not seen in any other infection group (FIG. 17). The enhanced level is maintained until day 6 p.i. On day 9 p.i., NOS II expressing cells are detected in greater quantities in all other treatment groups. At all days there does not appear to be a difference among control rVSVs and VSVwt at any time point.

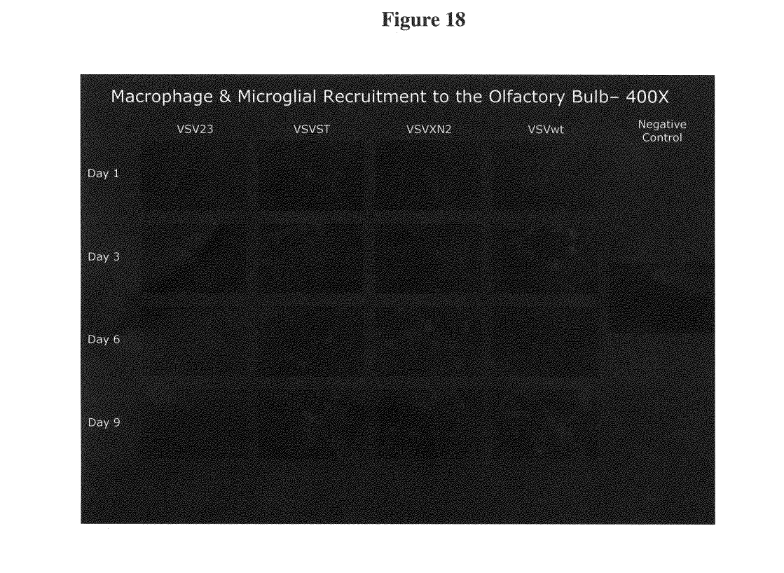

Macrophages and microglial cells are detected at increasing levels in all infection groups on days 1, 3, and 6 p.i. At most time points responses are similar, but on day 6 p.i. detection of CD11 b expressing cells appears to be decreased in VSV23 infected animals compared to other groups. On day 9 p.i., there is no detection of CD11b expressing cells in VSV23 infected animals (FIG. 18).

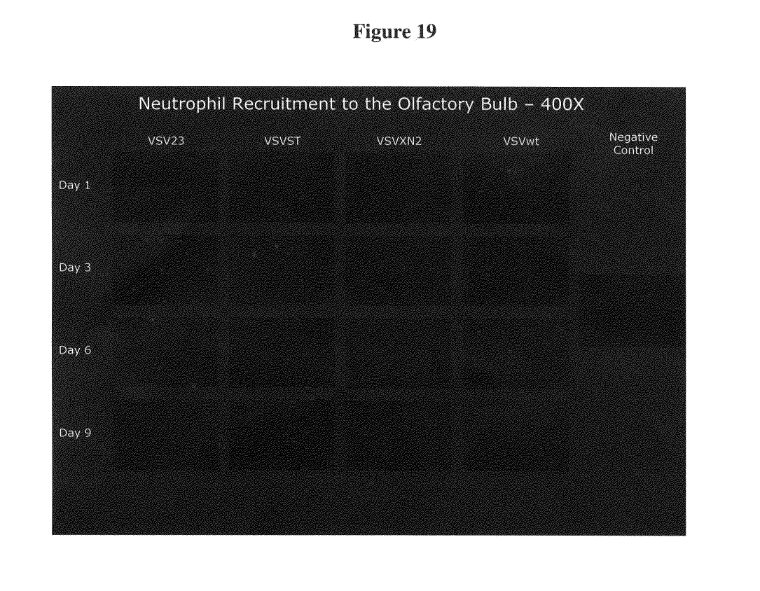

Neutrophils are detected in all infections at days 3 and 6 p.i., with more robust detection on day 6 p.i. There is a low level neutrophil response in VSVwt infected animals at day 1 p.i. No neutrophils are detected in any infection at day 9 p.i. (FIG. 19).

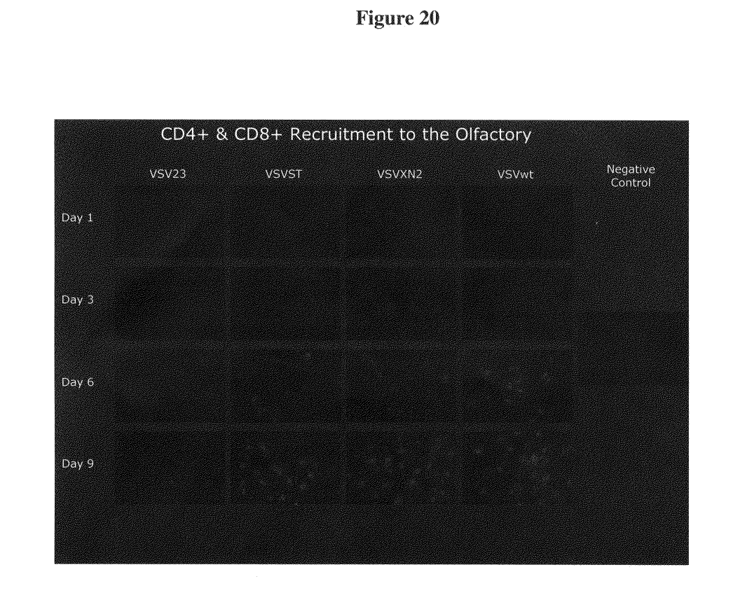

CD4⁺ and CD8⁺: CD4⁺ and CD8⁺T cells are detected at low levels in the olfactory bulb at day 6 p.i. in all infection groups. On day 9 p.i., all infection groups except VSV23 exhibit strong T cell responses. VSV23 induced T cell responses remain at levels similar to those seen on day 6 p.i. (FIG. 20).

Upregulation of NOS II expressing cells is in line with expectations. Greiss assay data indicated that by day 3 p.i. significantly greater levels of NO are present in VSV23 infected animals. Increased levels of NOS II expressing cells between days 1 and 3 p.i. are hypothesized to be responsible for the increase in NO levels. This hypothesis is further supported by the lack of increase in NOS I and NOS III expressing cells. It is interesting to note that while previous studies have indicated a role for NOS III in the immune response to VSV infection, alterations in NOS III expressing cells were not detected in this study.

vIL23 does not appear to induce an enhanced macrophage or microglial cell recruitment during infection in the CNS. It is likely that while vIL23 does not enhance recruitment, it does enhance cells antiviral activity through upregulation of NOS II. The decrease in CD11β cell detection at days 6 and 9 p.i. is likely due to the successful clearance of the virus. This hypothesis is supported by previous plaque assay data from the CNS of infected animals. At higher doses (1×10⁶), microglial and macrophage responses similar to those seen in control viral infections would be expected.

Enhanced recruitment of neutrophils was not seen in VSV23 infected animals. Neutrophil recruitment begins as early as 12 hours p.i. and peaks at 36 hours p.i. While neutrophils were not seen at day 1 p.i. in the olfactory bulb, they were seen at points of infiltration in other brain regions. It is hypothesized again that while vIL23 does not enhance recruitment it does enhance cells antiviral activity through upregulation of NOS II. Changes in neutrophils recruitment are not expected to be seen at higher doses; however, these studies are currently being conducted to attempt to disprove this hypothesis.

VSV23 infection does not induce significant CD4⁺ and CD8⁺T cell responses. It is hypothesized that rapid clearance of VSV23 from the CNS by the innate immune responses results in decreased recruitment of cells associated with the adaptive immune responses. Other components of the adaptive response such as antibody production and memory responses have not been shown to be decreased in VSV23 infection. In the event that VSV23 is able to withstand the innate immune responses, it is hypothesized that there would be robust T cell responses in the CNS comparable to those seen in control viruses.

### Example 12

### VSV23 Replicates in Tumor cells in vitro and Induces Apoptosis, Indicative of Killing the Tumor Cells

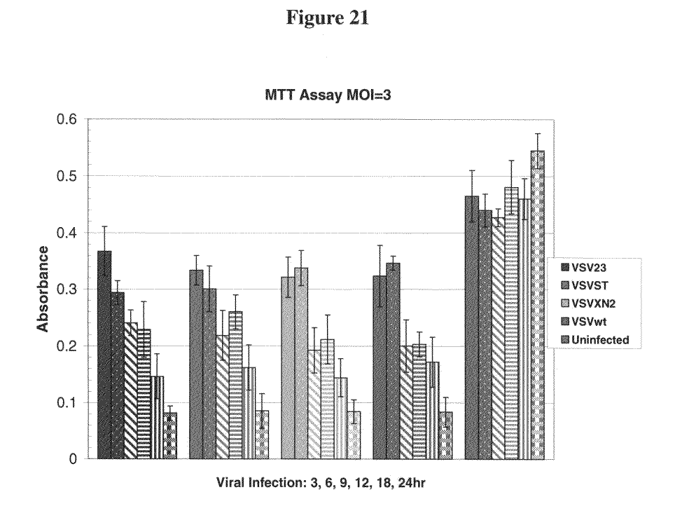

VSV23 can replicate in tumor cells and can induce killing of the tumor cells. The panel of viruses were used in an in vitro growth study in a breast cancer cell line (JC cells) and in an assay of apoptosis, the loss of mitochondrial potential (termed the MTT assay). VSV23 performed identically to the other panel members (FIG. 21) indicating it is not attenuated in its ability to destroy tumor cells.

VSV23 replicates as well in tumor cells as VSVwt or VSVXN2 viruses. VSV infection of susceptible cells rapidly leads to apoptosis due to both blockade of the nuclear pore complex and also to direct interactions with the mitochondria (Ahmed et al., “Ability of the Matrix Protein of Vesicular Stomatitis Virus to Suppress Beta Interferon Gene Expression is Genetically Correlated with the Inhibition of Host RNA and Protein Synthesis,” J Virol 77(8):4646-57 (2003); Gaddy et al., “Vesicular Stomatitis Viruses Expressing Wild-type or Mutant M Proteins Activate Apoptosis Through Distinct Pathways,” J Virol 79(7):4170-9 (2005); Lyles et al., “Potency of Wild-type and Temperature Sensitive Vesicular Stomatitis Virus Matrix Protein in the Inhibition of Host-directed Gene Expression,” Virology 225(1):172-80 (1996), which are hereby incorporated by reference in their entirety). The ability of VSV23 to induce apoptosis in a murine breast cancer cell line, JC cells (Capone et al., “Immunotherapy in a Spontaneously Developed Murine Mammary Carcinoma with Syngeneic Monoclonal Antibody,” Cancer Immunol Immunother 25(2):93-9 (1987), which is hereby incorporated by reference in its entirety), was tested in vitro. As is shown in FIG. 21, VSV23 rapidly induces depolarization of the mitochondrion which results in the inability of the infected cells to convert the MTT substrate to a colored product.

In vitro VSV23 Infection of Tumor Cells

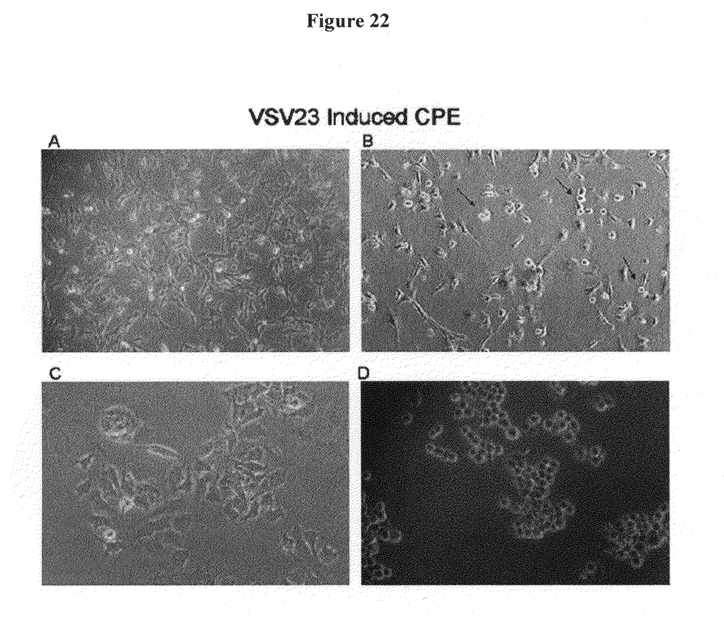

A major advantage of using VSV as a cancer treatment is the ability of the virus to infect a wide variety of tumor cells. In order to determine if VSV23 maintains this capacity, BHK21 cells were infected with VSV23 then virally infected supernatant was transferred to L929. Virally infected supernatant from L292 cells was transferred to NB41β3 cells and incubated until initial signs of CPE were noted and photographed (FIG. 22). RT-PCR and western blot analysis detected VSV M mRNA and VSV G and M proteins in VSV23-infected cell lysates. Taken together, it is shown that CPE in tumors, in vitro, was associated with VSV23 infection. The experiment was repeated with control viruses in order to determine their suitability for future experiments with similar results.

In vivo JC Tumor Treatment

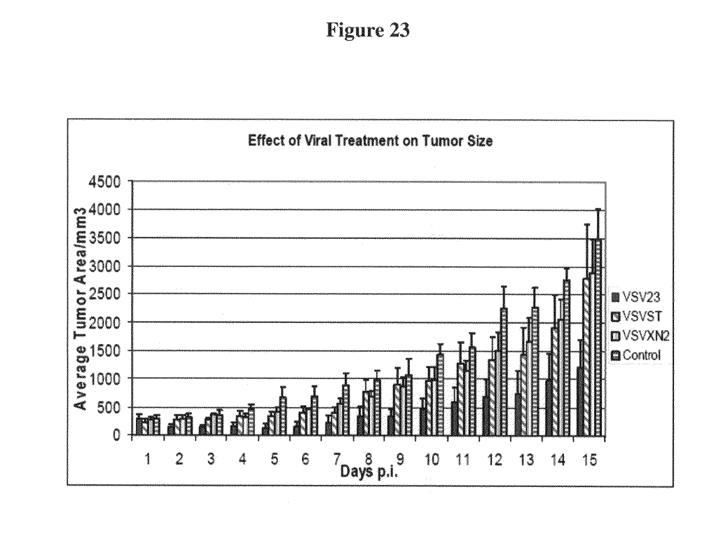

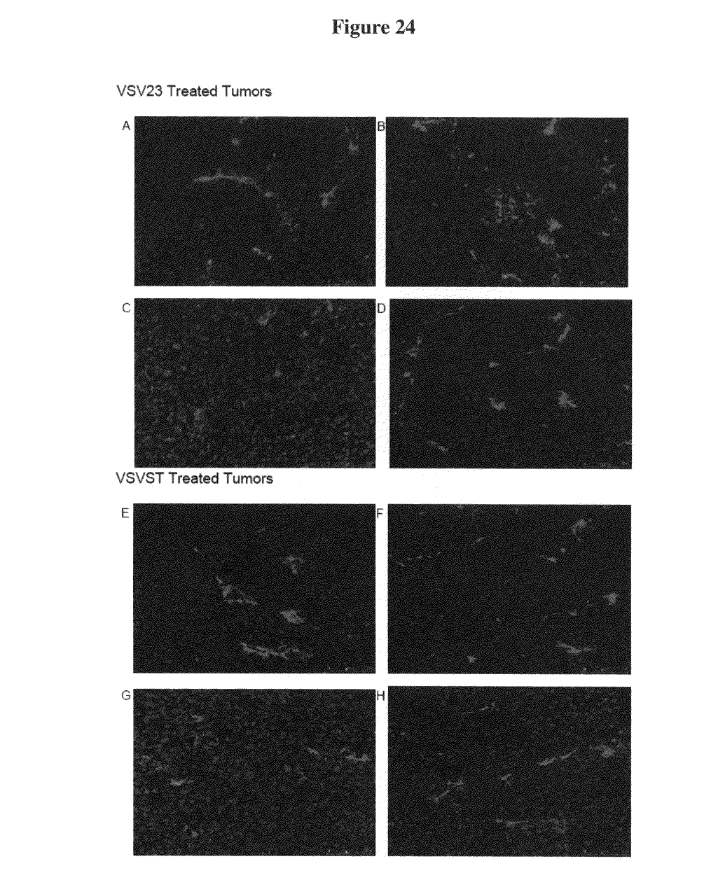

To test whether the oncolytic capacity of VSV23 remained intact in vivo and whether or not it was enhanced by the expression of IL-23, solid JC tumors were treated with VSV23 and the control viruses, VSVST and VSVXN2. Tumors treated with VSV23 exhibited a reduction in tumor size through the first 6 days of monitoring after treatment was initiated (FIG. 23). The average size of VSV23 treated tumors began to increase beyond the original measurement 8 days after treatment was initiated. In one case, a tumor was reduced to a size that could not be measured, however this near complete remission lasted only 2 days. Tumors treated with control viruses exhibited decreased growth rates compared to mock treated tumors during the first ten days after treatment; however they did not decrease in size from the initial measurement. By the end of the 14 day monitoring period control virus treated tumors were of similar size to untreated tumors, while VSV23 infected tumors remained significantly smaller than untreated tumors (p<0.005). There were no cases of complete tumor regression detected in any of the treatment groups.

### Immuno-histochemical Analysis of VSV23 Treated Tumors

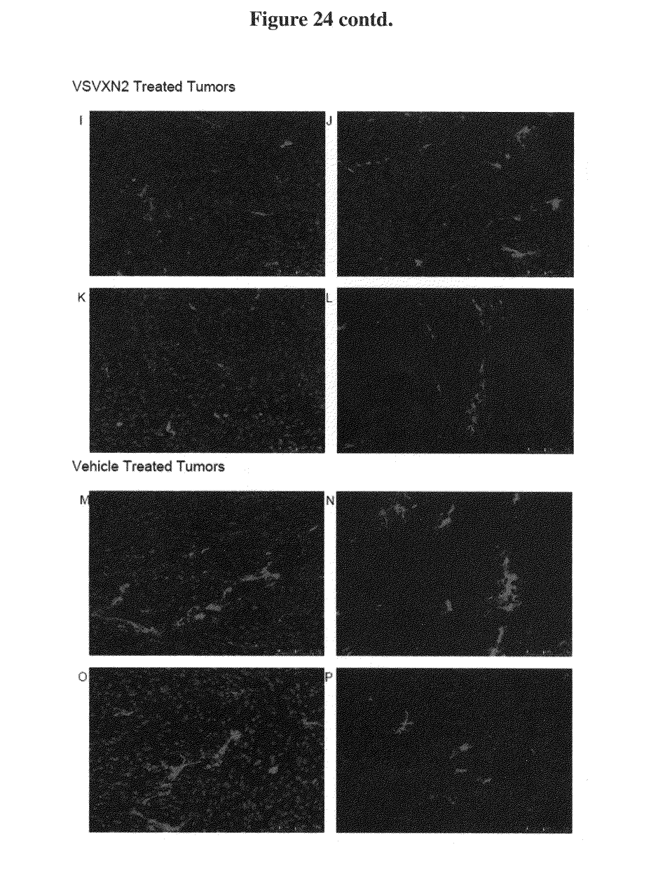

Immune responses against viral infection and tumor cells results in a variety of immune cell recruitment. Hypothetically, immune cell infiltration of tumors may be altered by VSV23 infection, due to the secretion of the cytokine. To test this, tumors were isolated 14 days after initiation of viral treatments. Tumors were sectioned and probed for macrophages, neutrophils, CD4β⁺, and CD8β⁺ cells. Analysis of slides using confocal microscopy indicated that all four cell types were recruited to tumors across the panel of viral treatments and mock infection (FIGS. 24 A-P). It was not possible to quantify infiltrating cells due to differences among tissue sections. VSV23-treated tumors appeared to have similar inflammatory cell responses when compared to tumors treated with control viruses and vehicle.

### Induction of Antitumor-Specific Cytolytic T Lymphocytes

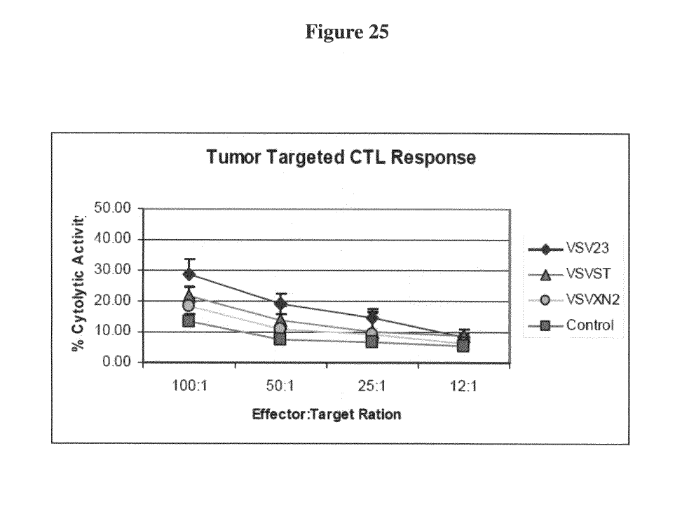

The ability of viruses to elicit host CTL responses which control infection and promote recovery is a hallmark of host acquired immunity to infection. In the case of tumors, CTL responses are induced at varying degrees of robustness and efficacy. In order to determine whether virally induced IL-23 (vIL-23) enhances CTL responses against tumor cells, splenocytes were harvested from tumor bearing animals 14 days after initiation of treatment. Splenocytes were then co-cultured with target JC cells and cell death was measured via a colorimetric assay. This experiment indicated that VSV23 was capable of inducing JC-specific memory CTLs and that the response is more robust than those induced in mock treated tumors (FIG. 25) P<0.05. Additionally, no statistical difference was detected among control viruses and mock treatment.

In summary, these experiments have demonstrated that VSV23 is highly immunogenic and not attenuated in vivo for eliciting innate NK cells, cellular (CD4 Th1 proliferating cells, CTLs) and humoral (neutralizing antibody) immune responses against VSV. However, it has also been shown that VSV23 is attenuated for causing viral encephalitis when administered by the crucial, sensitive intranasal route. Thus, it is highly likely to work well as a vaccine carrier of heterologous antigens and can be used for vaccination. Additionally, since VSV23 is attenuated for viral encephalitis associated with wildtype VSV, it is an ideal therapeutic candidate for use as a viral oncolytic agent.

Oncolytic tumor therapies are critical new agents in the treatment of cancers. The specificity of certain viruses for the infection of tumor cells, the ability to manipulate the genomes of viruses, and their capability to be used in conjunction with other viral therapies or traditional cancer treatments provide multiple avenues for study and improvement of treatment efficacy. The key issue is to balance the safety and immunogenicity of an attenuated or inactivated virus, such that the exposure of a host to attenuated viruses would elicit a potent immune response or oncolysis. Often times it is desirable that the viruses remain replication competent. Therefore, there is a need for safe and effective attenuation of VSV in order to minimize the risks associated with pathogenesis without jeopardizing its therapeutic potential.

VSV23 has been shown to be immunogenic in the periphery, attenuated for encephalitis in the CNS, and able to induce apoptosis in vitro and in vivo in a murine breast cancer model. These studies indicate that VSV23 has potential as a tumor treatment not only for breast cancer, but also in a great variety of tumors. All transformed cells that are identified as being deficient in interferon signaling and response are potential targets for VSV23 treatment. The known tropism of VSV in the CNS when administered intranasally also raises the possibility of using attenuated VSV23 as a treatment in inoperable brain tumors.

The limits of viral treatments are well known: host adaptive responses will eventually result in viral clearance and limit the time frame for effective tumor destruction. Previous work with viral oncolytics including VSV, has shown early promise in decreasing tumor size. Use in conjunction with other viral treatments (as well as more traditional treatments such as radiation and chemotherapy) may result in extended remission periods and a significantly improved quality of life.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A modified recombinant replicable vesiculovirus comprising vesiculovirus N, P, L proteins, and a replicable vesiculovirus genomic sense (−) RNA comprising an IL23 encoding nucleic acid molecule.

2. The IL23 encoding nucleic acid molecule according to claim 1, wherein the IL23 is a single chain molecule comprising the p40 and p19 subunits of IL23.

3. The modified recombinant vesiculovirus according to claim 1, wherein the IL23 encoding nucleic acid molecule is present in the replicable vesiculovirus genomic sense (−) RNA as:
(a) an insertion of an RNA complementary to the nucleic acid molecule which encodes the IL23 protein in a nonessential portion of said replicable vesiculovirus genomic sense (−) RNA, or
(b) a replacement of a nonessential portion of said replicable vesiculovirus genomic sense (−) RNA by an RNA complementary to the nucleic acid molecule which encodes the IL23 protein.

4. The vesiculovirus according to claim 3, wherein the vesiculovirus is vesicular stomatitis virus.

5. A host cell comprising the vesiculovirus according to claim 3.

6. The host cell according to claim 5 further comprising:
(a) a first recombinant nucleic acid molecule that can be transcribed to produce an RNA comprising a vesiculovirus antigenomic (+) RNA containing the vesiculovirus promoter for replication, in which a region of the RNA nonessential for replication of the vesiculovirus has been inserted into or replaced by the IL23 encoding RNA;
(b) a second recombinant nucleic acid molecule encoding a vesiculovirus N protein;
(c) a third recombinant nucleic acid molecule encoding a vesiculovirus L protein; and
(d) a fourth recombinant nucleic acid molecule encoding a vesiculovirus P protein.

7. The host cell according to claim 5 further comprising:
(a) a first DNA plasmid vector comprising the following operatively linked components:
(i) a bacteriophage RNA polymerase promoter;
(ii) a first DNA molecule that is transcribed in the cell to produce an RNA comprising (A) a vesiculovirus antigenomic (+) RNA containing the vesiculovirus promoter for replication, in which a region of the RNA nonessential for replication of the vesiculovirus has been inserted into or replaced by the IL23 encoding RNA, and (B) a ribozyme immediately downstream of said antigenomic (+) RNA, that cleaves at the 3′ terminus of the antigenomic RNA; and
(iii) a transcription termination signal for the RNA polymerase;
(b) a second DNA plasmid vector comprising the following operatively linked components:
(i) the bacteriophage RNA polymerase promoter;
(ii) a second DNA encoding a N protein of the vesiculovirus; and
(iii) a second transcription termination signal for the RNA polymerase;
(c) a third DNA plasmid vector comprising the following operatively linked components:
(i) the bacteriophage RNA polymerase promoter;
(ii) a third DNA encoding a P protein of the vesiculovirus; and
(iii) a third transcription termination signal for the RNA polymerase;
(d) a fourth DNA plasmid vector comprising the following operatively linked components:
(i) the bacteriophage RNA polymerase promoter;
(ii) a fourth DNA encoding a L protein of the vesiculovirus; and
(iii) a fourth transcription termination signal for the RNA polymerase; and
(e) a recombinant vaccinia virus comprising a nucleic acid molecule encoding the bacteriophage RNA polymerase, whereby in said cell the first DNA is transcribed to produce said RNA, the N, P, and L proteins and the bacteriophage RNA polymerase are expressed, and the modified recombinant replicable vesiculovirus is produced that has a genome that is the complement of said antigenomic RNA.

8. An isolated nucleic acid molecule which encodes the recombinant vesiculovirus according to claim 3.

9. A method of treating cancer in a subject, said method comprising:
selecting a subject with cancer and administering to the selected subject the recombinant vesiculovirus according to claim 3 under conditions effective to treat cancer.

10. The method according to claim 9, wherein the subject is a mammal.

11. The method according to claim 10, wherein the subject is a human.

12. The method according to claim 9, wherein the subject is avian.

13. The method according to claim 9, wherein the cancer is selected from the group consisting of melanoma, breast cancer, prostrate cancer, cervical cancer, hematological-associated cancer, and cancer caused due to defects in the tumor suppressor pathway.

14. The method according to claim 9, wherein said administering is carried out orally, parenterally, subcutaneously, intravenously, intramuscularly, intraperitoneally, by intranasal instillation, by application to mucous membranes, by direct contact to the cancer cells, by direct injection into the cancer cells, or by intratumoral injection to said subject.

15. The method according to claim 9, wherein the vesiculovirus is contained in a cell line infected with the virus and said administering is carried out intratumorally, intravenously, or intraperitoneally.

16. A composition comprising:
the vesiculovirus according to claim 3 and
a pharmaceutically acceptable carrier.

17. The vesiculovirus according to claim 3, wherein the replicable vesiculovirus genomic sense (−) RNA is further modified by:
(a) insertion of an RNA complementary to a nucleic acid molecule which encodes for a peptide or protein in a nonessential portion of said replicable vesiculovirus genomic sense (−) RNA, or
(b) replacement of a nonessential portion of said replicable vesiculovirus genomic sense (−) RNA by an RNA complementary to the nucleic acid molecule which encodes for a peptide or protein.

18. The vesiculovirus according to claim 17, wherein the peptide or protein is an immunogenic portion of a cancer specific or cancer associated antigen.

19. The vesiculovirus according to claim 17, wherein the peptide or protein is an immunogenic portion of an antigen of a pathogenic organism, wherein the pathogenic organism is selected from the group consisting of bacteria, virus, fungi, parasites, non-human pathogens, and human pathogens.

20. The vesiculovirus according to claim 17, wherein the vesiculovirus is vesicular stomatitis virus.

21. A host cell comprising the recombinant vesiculovirus according to claim 17.

22. An isolated nucleic acid molecule which encodes the recombinant vesiculovirus according to claim 17.

23. An immunogenic composition comprising:
the vesiculovirus according to claim 17 and
a pharmaceutically acceptable carrier.

24. A method for treating or preventing a disease or disorder mediated by a peptide or protein in a subject comprising:
selecting a subject in need of treatment or prevention of the disease or disorder and
administering to the selected subject the recombinant vesiculovirus according to claim 17 under conditions effective to induce an immune response against the peptide or protein.

25. The method according to claim 24, wherein the subject is a mammal.

26. The method according to claim 25, wherein the subject is a human.

27. The method according to claim 24, wherein the subject is avian.

28. The method according to claim 24, wherein said administering is carried out orally, parenterally, subcutaneously, intravenously, intramuscularly, intraperitoneally, by intranasal instillation, or by application to mucous membranes.

29. The method according to claim 24, wherein the vesiculovirus is contained in a cell line infected with the virus and said administering is carried out intratumorally, intravenously, subcutaneously, or intraperitoneally.

30. A recombinant, replicating and infectious vesicular stomatitis virus (VSV) particle comprising:
(a) a functional RNA dependent RNA polymerase (L);
(b) a vesiculovirus phosphoprotein (P);
(c) a vesiculovirus nucleocapsid (N);
(d) vesiculovirus protein selected from the group consisting of glycoprotein (G) and matrix (M);
(e) a 3′ non-coding RNA sequence;
(f) a 3′ to 5′ RNA coding sequence, which encodes the vesiculovirus L, P, N, and vesiculovirus protein required for assembly of budded infectious particles and including a nucleic acid molecule which encodes for IL23, wherein the nucleic acid molecule encoding IL23 is inserted at an intergenic junction; and
(g) a 5′ non-coding RNA sequence, wherein components (a) through (g) are from the same type of VSV.

31. The nucleic acid molecule according to claim 30, wherein the IL23 is a single chain molecule comprising the p40 and p19 subunits of IL23.
